(19) United States
(12) Patent Application Publication
(10) Pub. No.: US 2025/0046397 A1
(43) Pub. Date: Feb. 6, 2025

(54) GeneCull: Enabling High-Quality Gene Sequence Modeling via Evolution-Guided Data Pruning Criteria

(71) Applicant: Proteinea, Inc., Newton, MA (US)

(72) Inventors: Wafaa Ashraf SALAH-ELDIN, New Cairo (EG); Mohamed Ashraf ELKERDAWY, New Cairo (EG); Mohamed SOUDY, Cairo (EG); Ahmed EID ZOHEIR, Beni Suef (EG); Mohamed ELSHARKAWY, Newton, MA (US); Hosna ELTARRAS, Cairo (EG); Walid Moustafa, Cairo (EG); Abdulaziz ELGAMMAL, Giza (EG)

(73) Assignee: Proteinea, Inc., Newton, MA (US)

(21) Appl. No.: 18/793,780

(22) Filed: Aug. 3, 2024

Related U.S. Application Data
(60) Provisional application No. 63/532,137, filed on Aug. 11, 2023.
(60) Provisional application No. 63/530,548, filed on Aug. 3, 2023.

(51) Int. Cl. G16B 40/20; G16B 20/20

(52) U.S. Cl. CPC G16B 40/20; G16B 20/20

(57) ABSTRACT

A computer-implemented method is provided. The computer implemented method includes generating high-yield training datasets by excluding those genetic sequences that do not satisfy at least one abundance condition, at least one stability condition, at least one expression condition, and/or at least one translation efficiency condition. The computer-implemented method further includes training at least one model on the high-yield training datasets to generate high-yield genetic sequences.

### PRIORITY DATA

This application claims the benefit of (priority to) U.S. Provisional Application 63/530,548 filed on Aug. 3, 2023, entitled “GeneCull: Enabling High-Quality Gene Sequence Modeling via Evolution-Guided Data Pruning Criteria” (Attorney Docket No. PRTN1004USP01), and US Provisional Application 63/532,137 filed on Aug. 11, 2023, entitled “GeneCull: Enabling High-Quality Gene Sequence Modeling via Evolution-Guided Data Pruning Criteria” (Attorney Docket No. PRTN1004USP02).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Gene optimization techniques are at the forefront of scientific advancements in the fields of biotechnology and genetic engineering. These techniques, in the broad sense, are designed to enhance gene expression and protein production in specific host organisms through precise modifications of DNA sequences. Gene optimization arose as a data driven science, and often operates by exploring DNA sequence data rather than by testing preconceived models and hypotheses. Applications of gene optimization include biotechnological and biotherapeutic applications, in which researchers aim to achieve improved control over gene expression and enhance the yield of functional protein production. Despite notable advancements in this domain of gene optimization, significant challenges still exist in the heterologous expression of genes from one organism to another. These challenges lead to restricted protein yield, misfolding, and aggregation. Gene optimization is a process of particular importance in improving the expression and yield of recombinant proteins within specific host organisms. This optimization process targets specific regions within the plasmid—the extrachromosomal genetic vector that is being used to express the recombinant protein. These specific regions include the RBS (Ribosome Binding Site), CDSs (DNA Coding Sequences), and transcriptional terminators. Each of these regions plays a pivotal role in regulating gene expression and protein production.

The potency of gene optimization becomes particularly pronounced when considering recombinant protein production for therapeutic applications. Within this specialized area, addressing the challenges associated with cross-species gene expression is paramount. Such challenges include grappling with issues of inadequate protein yield, misfolding, and aggregation-factors that can significantly impede the efficacy of therapeutic interventions. Optimization of the RBS facilitates proper ribosome binding and translation initiation. Additionally, codon optimization of CDSs and signal peptides involves adapting the codons of the heterologous sequence to match the preferred codon usage of the host organism, leading to improved protein expression levels. Finally, proper optimization of the terminator region ensures accurate transcription termination, preventing unintended read-through or interference with neighboring genes. By meticulously tailoring the DNA sequence to align with the host organism, researchers can overcome challenges associated with heterologous gene expression thereby enhancing both the quality and quantity of the produced protein.

The significance of yield improvement within this therapeutic framework extends far beyond the laboratory bench. Indeed, the ability to control protein expression for yield quantity and quality improvement purposes has direct implications for the affordability and accessibility of critical/high-impact drugs. Optimized protein production stands as a potential cornerstone for driving down the costs of manufacturing, thereby increasing the accessibility of the biology in question. This facet resonates particularly and profoundly in the therapeutic landscape, where equitable access to effective treatments is of paramount importance.

The existing methods for gene optimization are classified as rule-based and machine learning-based. Rule-based methods hinge on predetermined criteria derived from factors such as codon usage frequency, mRNA secondary structure and stability, GC content, and restriction sites, often sourced from literature studies. However, these methods have significant limitations. Firstly, changes to synonymous codons can adversely impact the protein's structure, including its conformation, folding, stability, and post-translational modification sites. Additionally, relying solely on straightforward criteria like codon usage frequency and mRNA folding energy overlooks key biological factors influencing protein expression, such as sequence context, tRNA availability, ribosome pausing, mRNA degradation, and translation kinetics. Lastly, the optimal codons vary based on the host organism and protein type, making the generalization of rule-based techniques a source of inconsistency.

As a result of DNA sequence complexities and potential shortcomings of rule-based methods, analytical tools can be leveraged to support the discovery of protein expression, structure, and function. Unlike some algorithms, in which assumptions and domain expertise are hard coded, machine learning algorithms are designed to automatically detect patterns in data. Hence, machine learning algorithms are suited to data-driven sciences and, in particular, to gene optimization. However, the performance of machine learning algorithms can strongly depend on how the data are represented, that is, on how each variable (also called a feature) is computed.

Deep learning, a subdiscipline of machine learning, addresses the issue of data representation by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks can improve prediction accuracy by discovering relevant features of high complexity. The construction and training of deep neural networks have been enabled by the explosion of data, algorithmic advances, and substantial increases in computational capacity, particularly through the use of graphical processing units (GPUs).

Despite the transformative impact of machine learning-based techniques, particularly those harnessing the power of deep neural networks and specialized language models due to their ability to understand the underlying features in coding sequences and the relationship between host/protein-specific optimization, their practical success is often hindered by the prevalent issue of data sparsity, an issue that is heightened when the application of interest is yield optimization for therapeutics. In fact, the sparsity of yield-annotated data represents a significant obstacle to building deep learning-based filters. More particularly, given their data driven nature, deep learning methods are hindered by the sparsity of large-scale host/protein-specific data, or the high computational cost associated with multi-host/multi-protein data. Furthermore, deep learning methods are hindered by their limitation in covering long context lengths while retaining the relevance of distance tokens, which are of special importance in protein sequences. Finally, deep learning methods, in the best cases, produce a viable mapping of the protein sequence rather than an optimum one. These challenges hinder leveraging the aforementioned benefits of deep learning for gene optimization, in general, and codon optimization, in particular.

### SUMMARY

A computer-implemented method is provided. The computer implemented method includes generating high-yield training datasets by excluding those genetic sequences that do not satisfy at least one abundance condition, at least one stability condition, at least one expression condition, and/or at least one translation efficiency condition. The computer-implemented method further includes training at least one model on the high-yield training datasets to generate high-yield genetic sequences.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings.

FIG. 1 is a diagram showing one example of training a high-yield gene sequence generation model.

FIG. 2 is a flowchart showing one example operation of training a high-yield gene sequence generation model.

FIG. 3 is a flowchart showing another example operation of training a high-yield gene sequence generation model.

FIG. 4 is a diagram showing one example of a high-yield gene sequence generation system.

FIG. 5 is a diagram showing one example of a high-yield gene sequence prediction system.

FIG. 6 is a diagram showing one example generation of a high-yield training dataset.

FIG. 7 is a diagram showing another example generation of a high-yield training dataset.

FIG. 8 is a block diagram of a computer system that can be used to implement the technology disclosed.

FIGS. 9A-9B include a table and a chart that show the fold-change in yield/expression for five proteins that were each expressed in the HEK293 cell line.

FIGS. 10A-10B include a table and a chart that show the fold-change in yield/expression for three proteins that were each expressed in the Yeast Pichia cell line.

FIGS. 11A-11B include a table and a chart that show the fold-change in yield/expression for two proteins that were each expressed in the HEK293 cell line.

FIGS. 12A-12C include a table that shows the fold-change in yield/expression for two protein classes, each with several corresponding protein indices, that were each expressed in the HEK293 cell line.

FIG. 13 includes a table that shows the fold-change in yield/expression for two protein classes, each with several corresponding protein indices, that were each expressed in the CHO cell line.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Gene optimization techniques aim to improve gene expression and protein yield in a specific host organism via modifying the DNA sequence. Gene optimization is at the forefront of scientific advancements in the field of biotechnology and genetic engineering. Gene optimization, in general, and codon/CDSs optimization, in particular, are classified as either rule-based or deep learning-based methods. Rule-based methods employ predetermined criteria based on various factors to select ideal codons and enhance protein expression. The criteria typically include codon usage frequency, secondary structure, mRNA stability, GC content, motifs, and restriction sites, which are derived from literature studies. However, rule-based methods have significant drawbacks. For example, synonymous changes to the gene sequence can have negative effects on the protein's structure and function, such as altering its conformation, folding, stability, and post-translational modification sites. Secondly, simple criteria like codon usage frequency and mRNA folding energy overlook important biological factors that influence protein expression, such as sequence context, availability of transfer RNA (tRNA), ribosome pausing, mRNA degradation, and translation kinetics. Lastly, optimal codons vary depending on the host organism, protein type, and experimental conditions, leading to inconsistencies when different optimization methods are employed due to variations in data sources and algorithms. As a result of the aforementioned problems relating to rule-based methodology for gene optimization, researchers utilize deep learning-based methods in order to learn how to produce a host-specific codon sequence as an output given an amino acid sequence as an input.

The employment of deep learning-based methods brings out its own challenge of identifying the criteria for high-quality training data, enabling high-quality sequence generation, and identifying the mechanisms by which the generated data can be filtered into top sequences in terms of yield. In particular, the sparsity of yield-annotated data represents a significant obstacle to building deep learning-based filters. Such a challenge hinders leveraging the various benefits of deep learning for gene optimization, in general, and codon optimization, in particular. Therefore, despite the advancements in the domain of optimization, significant challenges still exist in the heterologous expression of genes from one organism in another, leading to restricted protein yield, misfolding, and aggregation, primarily attributed to disparities in the critical regions.

Disclosed is a deep learning-based framework that generates high-quality gene sequences, in general, and codon sequences, in particular, referred to as “GeneCull.” The GeneCull framework evades the challenges introduced via the utilization of deep learning advances by addressing the data sparsity limitations disabling the identification of quality metrics for both training data preprocessing as well as generated data filtration. The GeneCull framework seeks to evade these challenges by utilizing attainable criteria to denote yield quality, discussed below. Utilization of the aforementioned criteria enforces that protein abundance, mRNA abundance, protein stability, mRNA stability, stable/consistent expression, ubiquitous expression, and translation efficiency (as expressed via multiple criteria, a consensus of the criteria, or via each criterion solely, within predetermined thresholds) denotes an evolutionary gene preference per host. For example, genes with the highest consensus of abundance thresholds represent optimum genes with respect to the host and/or protein in question.

The GeneCull framework utilizes attainable criteria to prune training datasets by omitting sequences that do not satisfy the utilized criteria. Thus, the utility of the GeneCull framework translates to the generation of high-quality gene sequences corresponding to higher percentages of protein yield when compared to other frameworks.

In one example, the GeneCull framework is trained via the utilization of a neural network system. A neural network system processes the input representations as an input and generates the output representations as output. In some implementations, the neural network system utilized to train the GeneCull framework is at least one of a language model neural network, a sequence-to-sequence neural network, a variational autoencoder neural network, a generative adversarial neural network, a diffusion neural network, a recurrent neural network, an autoregressive neural network, an energy-based neural network, and a flow-based neural network. However, it is expressly contemplated that other neural network systems can be utilized as well.

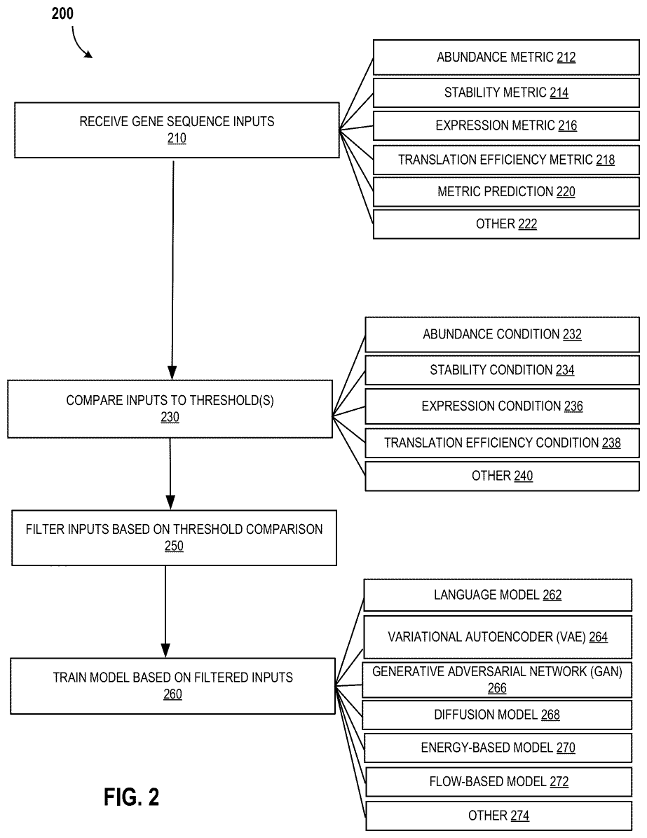

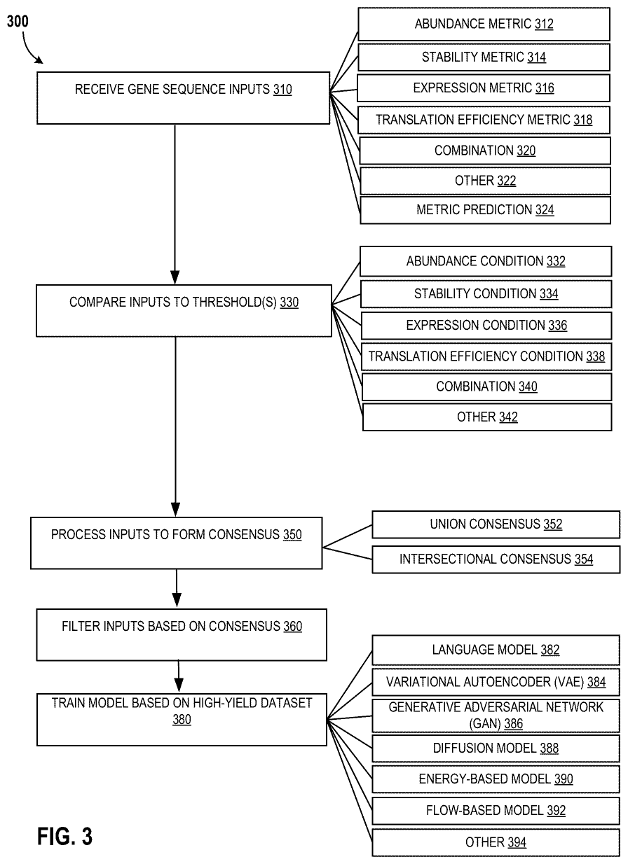

The application of the GeneCull framework can be verified across the four edit-prone regions in the plasmid: the promoter sequence, the ribosome binding site, the codon sequence, and terminators. Additionally, the GeneCull framework enables and validates the use of each criterion solely and collectively. As further discussed below, the values of each utilized criterion can be ground-truth (i.e., characterized in a unified experimental setting) or predicted (i.e., inferred via GeneCull regressors). In some examples, the GeneCull regressors can be trained utilizing the same methods described below with respect to FIGS. 2-3. However, in other examples, the GeneCull regressors can be trained in other ways as well.

### Abundance Conditions

The GeneCull framework utilizes, amongst other conditions, one or more abundance conditions to prune training datasets. As used herein, a “condition” is defined as a requirement that must be satisfied for a given input (e.g., a sequence) to be included in a filtered dataset (e.g., a training dataset). In particular, GeneCull prunes training datasets by omitting sequences that do not satisfy the one or more abundance conditions. In some examples, the abundance conditions can include mRNA abundance and/or protein abundance.

mRNA abundance refers to obtaining sequences with the highest mRNA abundance percentages (top N % or top-N sequences), where the abundance condition is decided upon visualizing the label range as well as dataset size. More particularly, mRNA abundance refers to the quantity of a specific mRNA molecule present in a cell at a given time. It is affected by the balance between two processes: transcription efficiency (i.e. generation) and mRNA stability (i.e. degradation). mRNA abundance can be determined by the thresholding of one or more associated metrics. As used herein, a “metric” is defined as a measurement unit for a respective condition, the value of which can be compared to an associated threshold. For example, one or more metrics can be compared to an associated threshold to determine mRNA abundance. These metrics are common normalization methods used to compare mRNA levels across different samples or genes. For instance, mRNA abundance can be determined by thresholding based on a Reads Per Kilobase Million (RPKM) metric. RPKM is a measure of gene expression commonly used in RNA sequencing. RPKM represents the number of reads mapped to a gene normalized by gene length and sequencing depth and takes into account both gene length and the total number of reads in an experiment. RPKM can be determined by the following equation:

$$RPKM = ( Number of reads mapped to a transcript ) / ( Length of the transcript in kilobases * Total number of reads in millions ) .$$

In another example, mRNA abundance can be determined by thresholding based on a Fragments Per Kilobase Million (FPKM) metric. FPKM bears many similarities to RPKM but is used when the sequencing technology generates short fragments (rather than full-length reads). Hence, FPKM is utilized for normalizing paired-end reading, right and left, of the fragment. FPKM is also used in RNA-sequencing (RNA-seq) analysis, and provides a normalized measure of gene expression, considering fragment length and sequencing depth. FPKM can be determined by the following equation:

$$FPKM = ( Number of fragments mapped to a transcript ) / ( Length of the transcript in kilobases * Total number of fragments in millions ) .$$

In another example, mRNA abundance can be determined by thresholding based on a Transcripts Per Million (TPM) metric. TPM is a measurement of gene expression commonly used in RNA-seq analysis. TPM represents the relative abundance of a transcript in a sample by normalizing transcript counts not only by transcript length and sequencing depth, but also by the proportion of each transcript within the sample. TPM provides a standardized way to compare gene expression levels between different samples and becomes an accurate method for comparing data from different samples. TPM can be determined by the following equation:

$$TPM = ( Number of reads mapped to a transcript / Length of the transcript in kilobases ) / Sum of all normalized transcript counts .$$

Of course, it is expressly contemplated that mRNA abundance can be determined by thresholding based on two or more of the above-noted metrics (e.g., by the utilization of a union consensus or an intersectional consensus, discussed below). For example, mRNA abundance can be determined by thresholding based on RPKM, FPKM, and/or TPM metrics. Additionally, it is expressly contemplated that other metrics can be utilized to determine mRNA abundance as well.

Protein abundance refers to obtaining sequences with the highest protein abundance percentages (top N % or top-N sequences), where the selected threshold is decided upon visualizing the label range as well as dataset size. Protein abundance is indicative of the concentration of a specific protein in a cell. It is affected by the balance between two processes: translation efficiency (i.e., generation) and protein stability (i.e., degradation). Protein abundance can be determined by thresholding based on one or more associated metrics. For example, protein abundance can be determined by thresholding based on a Protein Per Million (PPM) metric. PPM is a metric used to quantify the abundance of a particular protein, often in mass spectrometry. PPM reflects the number of a protein molecule relative to the total number of one million molecules in the sample.

In one example, protein abundance can be determined by thresholding based on a titer metric. A titer metric refers to the concentration of a protein in a solution, commonly used in the context of proteins in solutions, but can also be a ratio of volume-to-volume, weight-to-volume, or weight-to-weight. One example of a titer metric is milligrams-per-liter (mg/L). An mg/L metric is a measure of concentration by weight of a protein (milligrams) per unit of volume (liters). Although mg/L is used herein as an example, it is expressly contemplated that any other concentration measurements can be utilized as a titer metric as well. For example, a molarity (M) metric can also be utilized, which refers to the concentration of a protein in terms of moles-per-liter (mol/L). In another example, a protein concentration metric can be utilized as the titer metric as well.

Of course, it is expressly contemplated that protein abundance can be determined by thresholding based on two or more of the above-noted metrics (e.g., by the utilization of a union consensus or an intersectional consensus, discussed below). For example, protein abundance can be determined by thresholding based on PPM, yield, and/or titer metrics. Additionally, it is expressly contemplated that other metrics can be utilized to determine protein abundance as well.

### Stability Conditions

The GeneCull framework also utilizes, amongst other conditions, one or more stability conditions to prune training datasets. In particular, GeneCull prunes training datasets by omitting sequences that do not satisfy the one or more stability conditions. In some examples, the stability conditions can include mRNA stability and/or protein stability.

mRNA stability refers to obtaining sequences with the highest mRNA stability values, where the selected threshold is decided upon visualizing the label range as well as dataset size. mRNA stability is indicative of the lifespan of an mRNA molecule before it is degraded. mRNA stability influences overall mRNA abundance and, consequently, protein production. The stability of mRNA molecules is influenced by various factors, including the presence of specific sequences in the mRNA (e.g., AU-rich elements) and the action of RNA-binding proteins and microRNAs that can promote or inhibit mRNA degradation. mRNA stability can be determined by thresholding based on one or more associated metrics. For example, mRNA stability can be determined by thresholding based on an mRNA Half-Life metric. mRNA half-life refers to the time it takes for half of the mRNA molecules in a cell to be degraded. mRNA half-life is an important determinant of expression levels. Short-lived mRNAs are rapidly degraded, leading to lower protein production, while long-lived mRNAs can persist for a more extended period of time, resulting in higher protein levels. mRNA half-life can be measured experimentally through RNA decay assays.

In another example, mRNA stability can be determined by thresholding based on degradation rate metrics. mRNA degradation rate refers to the rate at which mRNA molecules are broken down, often expressed as a degradation constant (k). It can be determined by analyzing the decay kinetics of mRNA over time.

Of course, it is expressly contemplated that mRNA stability can be determined by thresholding based on two or more of the above-noted metrics (e.g., by the utilization of a union consensus or an intersectional consensus, discussed below). For example, mRNA stability can be determined by thresholding based on mRNA half-life and degradation rate metrics. Additionally, it is expressly contemplated that other metrics can be utilized to determine mRNA stability as well, such as Average Unpaired Probability (AUP) and/or Differentially Expressed Gene (DEG) score.

Protein stability refers to obtaining sequences with the highest protein stability values, where the selected threshold is decided upon visualizing the label range as well as dataset size. Protein stability is indicative of the lifespan of a protein molecule before it's degraded, and therefore influences protein abundance in the cell. Protein stability can be determined by thresholding based on one or more associated metrics. For example, protein stability can be determined by thresholding based on a Protein Half-Life metric. Protein half-life refers to the time it takes for half of the protein molecules in a cell to be degraded. Protein half-life can be measured using protein turnover assays.

In another example, protein stability can be determined by thresholding based on degradation rate metrics. Protein degradation rate refers to the rate at which protein molecules are broken down, often expressed as a degradation constant (k). Protein degradation rate is determined by analyzing protein decay kinetics over time.

Additionally, it is expressly contemplated that protein stability can be determined by thresholding based on two or more of the above-noted metrics. For example, protein stability can be determined by thresholding based on protein half-life and protein degradation rate metrics. Further, it is expressly contemplated that other metrics can be utilized to determine protein stability as well.

### Expression Conditions

The GeneCull framework also utilizes, amongst other conditions, one or more expression conditions to prune training datasets. In particular, GeneCull prunes training datasets by omitting sequences that do not satisfy the one or more expression conditions. In some examples, the expression conditions can include stable/consistent expression and/or ubiquitous expression.

Stable/consistent expression refers to genes that are expressed at stable, consistent levels across different cell types and conditions. Stable/consistent expression can be determined by the thresholding of one or more associated measurements. For example, stable/consistent expression can be determined by the thresholding of Housekeeping Genes metrics. Housekeeping genes are essential for cellular existence regardless of their specific function in the tissue or organisms, and typically have high abundance and consistent expression. These genes encode proteins that are involved in fundamental cellular processes such as DNA replication and repair, cellular metabolism, cell structure, and function. Housekeeping genes are typically stable in their mRNA levels and/or protein levels, which can be measured by, for example, the aforementioned mRNA abundance and protein abundance. Utilizing housekeeping genes provides the language model with the codon composition and codon pattern, which reflects the highly abundant, stably expressed, and/or consistently expressed proteins. Additionally, it is expressly contemplated that other metrics can be utilized to determine stable/consistent expression as well.

In another example, stable/consistent expression can be determined by the thresholding of Collagen metrics. Collagen is the most abundant protein in most mammals. Moreover, collagen is widely used in fingerprinting of historical samples due to its stability and abundance. Accordingly, a collection of collagen gene sequences is utilized, which are annotated with their expression to train a language model to generate highly abundant and stable genes. Additionally, it is expressly contemplated that other metrics can be utilized to determine stable/consistent expression as well.

Ubiquitous expression bears many similarities to stable/consistent expression and refers to genes that are expressed in the majority of the cells of an organism. Ubiquitous expression can be determined by the thresholding of one or more associated measurements. For example, ubiquitous expression can be determined by the thresholding of Collagen metrics. Collagen is the most abundant protein in most mammals. Moreover, collagen is widely used in fingerprinting of historical samples due to its stability and abundance. Accordingly, a collection of collagen gene sequences is utilized, which are annotated with their expression to train a language model to generate highly abundant and stable genes. Additionally, it is expressly contemplated that other metrics can be utilized to determine ubiquitous expression as well.

In another example, ubiquitous expression can be determined by the thresholding of Housekeeping Genes metrics. Housekeeping genes are essential for cellular existence regardless of their specific function in the tissue or organisms, and typically have high abundance and consistent expression. These genes encode proteins that are involved in fundamental cellular processes such as DNA replication and repair, cellular metabolism, cell structure, and function. Housekeeping genes are typically stable in their mRNA levels and/or protein levels, which can be measured by, for example, the aforementioned mRNA abundance and protein abundance. Utilizing housekeeping genes provides the language model with the codon composition and codon pattern, which reflects the highly abundant, stably expressed, and/or consistently expressed proteins. Additionally, it is expressly contemplated that other metrics can be utilized to determine ubiquitous expression as well. Translation Efficiency Conditions

The GeneCull framework utilizes, amongst other conditions, a translation efficiency condition to prune training datasets. Thresholding based on translation efficiency conditions refers to obtaining sequences with the highest translational efficiency percentages (top N % or top-N sequences), where the selected threshold is decided upon visualizing the label range as well as dataset size. Translation efficiency is indicative of how efficiently mRNA is converted into protein. It is influenced by factors like mRNA codon bias, tRNA availability, ribosome availability, and translation initiation factors. GeneCull prunes training datasets by omitting sequences that do not satisfy the translation efficiency condition. Translation efficiency can be determined by thresholding based on one or more associated metrics. For example, translation efficiency can be determined by thresholding based on a Protein Per Transcript (PPT) metric. PPT is a metric used to quantify the abundance of a protein relative to the level of its corresponding mRNA transcript. The PPT value is calculated by dividing the protein abundance (measured, for example, by mass spectrometry) by the corresponding mRNA abundance (measured, for example, by RNA sequencing). PPT is a valuable metric as it provides insights into the efficiency of translation from mRNA to protein as it maps the relation between mRNA expression levels and protein levels, determining the protein turnover of an mRNA transcript.

In another example, translation efficiency can be determined by thresholding based on a Protein-to-mRNA Ratio (PTR) metric. In addition to quantifying the abundance of a protein relative to the level of its corresponding mRNA transcript, such as that with PPT, PTR accounts for both protein stability and mRNA stability.

In another example, translation efficiency can be determined by thresholding based on a Ribosomal Profiling metric. Ribosomal profiling measures ribosome occupancy on mRNA to assess translation rates. This technique provides a snapshot of ribosome positions on mRNA, giving insights into translation rates and ribosome density. Ribosomal profiling involves sequencing ribosome-protected mRNA fragments to determine which mRNAs are being actively translated, and how efficiently.

Additionally, it is expressly contemplated that translation efficiency can be determined by thresholding based on two or more of the above-noted metrics (e.g., by the utilization of a union consensus or an intersectional consensus, discussed below). For example, translation efficiency can be determined by thresholding based on PPT, PTR, and/or ribosomal profiling metrics. Additionally, it is expressly contemplated that other metrics can be utilized to determine translation efficiency as well.

### Intersectional Consensus

As further described below, an optional component of the GeneCull framework is the utilization of consensus thresholding to further prune training datasets. Consensus thresholding can include, for example, forming an intersectional consensus. An intersectional consensus is a thresholding step in which only the sequences that exist within each of the utilized conditions and/or associated thresholds proceed. In this example, sequences that do not overlap between each utilized condition and/or threshold are omitted.

An intersectional consensus can be utilized at two levels of training dataset generation. The first level in which an intersectional consensus can be utilized is at the metric level for each utilized yield-quality condition. At the metric level, the outputs from applying the intersectional consensus on a set of sequence inputs are the sequences that satisfy each of the utilized thresholds of the given condition. For instance, an abundance condition indicating mRNA abundance can be utilized for training dataset generation, in which an intersectional consensus can be applied at the metric level to a set of gene sequence inputs. In this instance, sequences that overlap between each utilized threshold are determined to have mRNA abundance and proceed, and sequences that do not overlap between each utilized threshold are omitted. The utilized thresholds for mRNA abundance can be, for example, at least two of an RPKM-based threshold, an FPKM-based threshold, and/or a TPM-based threshold.

By way of example, if an abundance condition utilizing an RPKM-based threshold, an FPKM-based threshold, and a TPM-based threshold is applied to an input of one hundred sequences, and it is determined that ten sequences overlap between each of the utilized RPKM-based threshold, FPKM-based threshold, and TPM-based threshold, the resulting output from the intersectional consensus would be the ten sequences, indicating that each of the ten sequences has mRNA abundance.

While one hundred sequences are used in the above example, one skilled in the art would appreciate that this is merely for the purpose of explanation, and a smaller or larger number of sequences can also be used as an input. For example, the number of sequences used as an input can be in the range of thousands, ten thousands, hundred thousands, or millions.

The second level in which an intersectional consensus can be utilized is at the condition level. At the condition level, the outputs from applying the intersectional consensus on a set of sequence inputs are the sequences that overlap between each utilized yield-quality condition. For instance, at least two of an abundance condition, a stability condition, an expression condition, and/or a translation efficiency condition can be utilized for generating the training dataset, in which an intersectional consensus can be applied at the condition level to the gene sequence inputs. In this instance, sequences that overlap between each of the utilized conditions proceed, and sequences that do not overlap between each utilized condition are omitted.

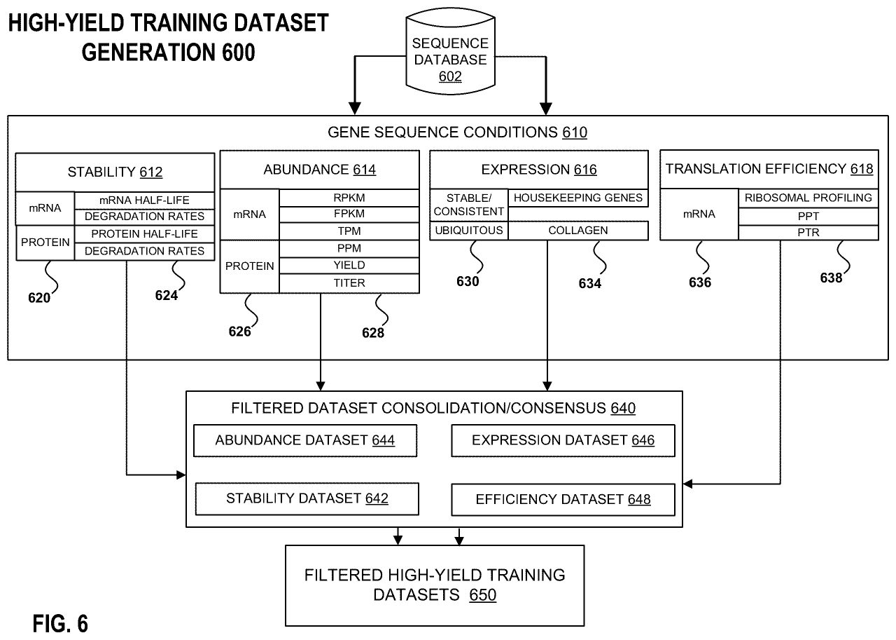

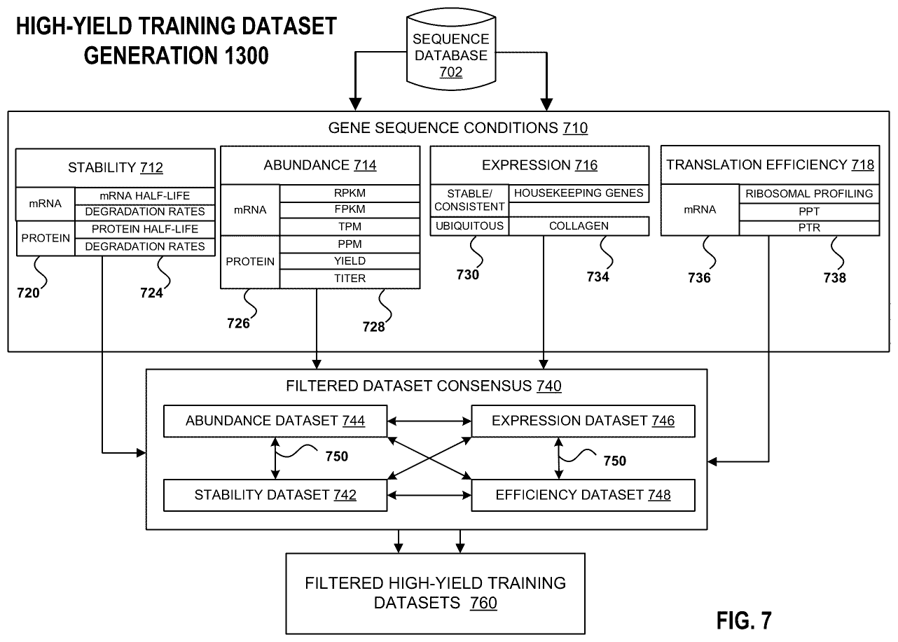

In another example, an intersectional consensus can be formed at the condition level for at least two sub-conditions of a given condition. For instance, at least two of an abundance condition indicating mRNA abundance, and an abundance condition indicating protein abundance can be utilized for generating the training dataset, in which an intersectional consensus can be applied at the condition level to the gene sequence inputs. In this instance, sequences that overlap between each of the utilized conditions (e.g., mRNA abundance and protein abundance) proceed, and sequences that do not overlap between each utilized condition are omitted. Of course, it is expressly contemplated that an intersectional consensus can be formed at the condition level for other sub-conditions as well, such as any of those set forth below with respect to FIGS. 6-7.

By way of example, if an abundance condition, a stability condition, and an expression condition are applied to a dataset of one thousand gene sequences, and it is determined that three hundred sequences overlap between each of the utilized abundance condition, stability condition, and expression condition, the resulting output from the intersectional consensus would be three hundred sequences, indicating that each of the three hundred sequences overlap between each of the abundance condition, stability condition, and expression condition.

While one thousand gene sequences are used in the above example, one skilled in the art would appreciate that this is merely for the purpose of explanation, and a smaller or larger number of sequences can also be used as an input. For example, the number of sequences used can be in the range of thousands, ten thousands, hundred thousands, or millions.

Additionally, it is expressly contemplated that the intersectional consensus can occur at one or both above-noted levels of high-yield training dataset generation. For example, the intersectional consensus can be utilized at only the metric level. In another example, the intersectional consensus can be utilized at only the condition level. In another example, the intersectional consensus can be utilized at both the metric level and the condition level. However, as the GeneCull framework supports and validates the use of each condition solely, the intersectional consensus component remains optional. Union Consensus

As further described below, another optional component of the GeneCull framework is the utilization of a union consensus. A union consensus is a thresholding step in which the sequences that exist within at least two of the utilized conditions and/or associated thresholds proceed. In this example, sequences that do not merge the at least two utilized conditions and/or thresholds are omitted.

A union consensus can be utilized at two levels of training dataset generation. The first level in which a union consensus can be utilized is at the metric level for each utilized yield-quality condition. At the metric level, the outputs from applying the union consensus on a set of sequence inputs are the sequences that merge at least two of the utilized thresholds of the given condition. For instance, an abundance condition indicating mRNA abundance can be utilized for training dataset generation, in which a union consensus can be applied at the metric level to a set of gene sequence inputs. In this instance, sequences that merge at least two of the utilized thresholds are determined to have mRNA abundance and proceed, and sequences that do not merge at least two of the utilized thresholds are omitted. The utilized thresholds for mRNA abundance can be, for example, an RPKM-based threshold, an FPKM-based threshold, and/or a TPM-based threshold.

By way of example, if an abundance condition utilizing an RPKM-based threshold and an FPKM-based threshold is applied to an input of one hundred sequences, and it is determined that twenty sequences merge the RPKM-based threshold and ten different sequences merge the FPKM-based threshold, the resulting output from the union consensus would be thirty sequences, indicating that each of the thirty sequences has mRNA abundance.

While one hundred sequences are used in the above example, one skilled in the art would appreciate that this is merely for the purpose of explanation, and a smaller or larger number of sequences can also be used as an input. For example, the number of sequences used as an input can be in the range of thousands, ten thousands, hundred thousands, or millions.

The second level in which a union consensus can be utilized is at the condition level. At the condition level, the outputs from applying the union consensus on a set of sequence inputs are the sequences that merge at least two of the utilized yield-quality conditions. For instance, at least two of an abundance condition, a stability condition, an expression condition, and/or a translation efficiency condition can be utilized for generating the training dataset, in which a union consensus can be applied at the condition level to the gene sequence inputs. In this instance, sequences that merge at least two utilized conditions proceed, and sequences that do not merge at least two utilized conditions are omitted.

In another example, a union consensus can be formed at the condition level for at least two sub-conditions of a given condition. For instance, at least two of an abundance condition indicating mRNA abundance, and an abundance condition indicating protein abundance can be utilized for generating the training dataset, in which a union consensus can be applied at the condition level to the gene sequence inputs. In this instance, sequences that merge at least two of the utilized conditions (e.g., mRNA abundance and protein abundance) proceed, and sequences that do not merge at least two utilized conditions are omitted. Of course, it is expressly contemplated that a union consensus can be formed at the condition level for other sub-conditions as well, such as any of those set forth below with respect to FIGS. 6-7.

By way of example, if an abundance condition, a stability condition, and an expression condition are applied to a dataset of one thousand gene sequences, and it is determined that one hundred sequences merge the abundance condition, three hundred different sequences merge the stability condition, and fifty different sequences merge the expression condition, the resulting output from the union consensus would be four hundred and fifty sequences, indicating that each of the four hundred and fifty sequences merge at least two of the abundance condition, stability condition, and expression condition.

While one thousand gene sequences are used in the above example, one skilled in the art would appreciate that this is merely for the purpose of explanation, and a smaller or larger number of sequences can also be used as an input. For example, the number of sequences used can be in the range of thousands, ten thousands, hundred thousands, or millions.

Additionally, it is expressly contemplated that the union consensus can occur at one or both above-noted levels of high-yield training dataset generation. For example, the union consensus can be utilized at only the metric level. In another example, the union consensus can be utilized at only the condition level. In another example, the union consensus can be utilized at both the metric level and the condition level.

### High-Yield Gene Sequences

Aspects of the present disclosure discuss various methods and embodiments of generating high-yield training datasets and high-yield gene sequence predictions. As used herein, “high-yield” is intended to mean gene sequences that satisfy at least one predetermined condition that denotes yield-quality. For example, high-yield may refer to any gene sequence that satisfies one or more of an abundance condition, stability condition, expression condition, and translation efficiency condition, discussed above and described in more detail below with respect to FIGS. 6-7. More specifically, “high-yield” may refer to any gene sequence that satisfies an associated threshold or thresholds for a given yield-quality condition. Examples of such thresholds are discussed in more detail below with respect to FIGS. 6-7.

### Codon Sequences

Aspects of the present disclosure discuss various methods and embodiments of utilizing gene sequences and generating high-yield gene sequence predictions. As discussed herein, it is expressly contemplated that utilizing gene sequences and/or generating high-yield gene sequence predictions can include, for example, utilizing codon sequences and/or generating high-yield codon sequence predictions.

IMPLEMENTATIONS

In this disclosure, the example embodiments may use various machine learning models for the codon sequence optimization and the high-yield codon generation problems described above. As will be described in more detail, the machine learning models may require sample data (also referred to as training data) to make predictions or decisions. In the description that follows, various implementations of the disclosed technology are described with reference to the following figures.

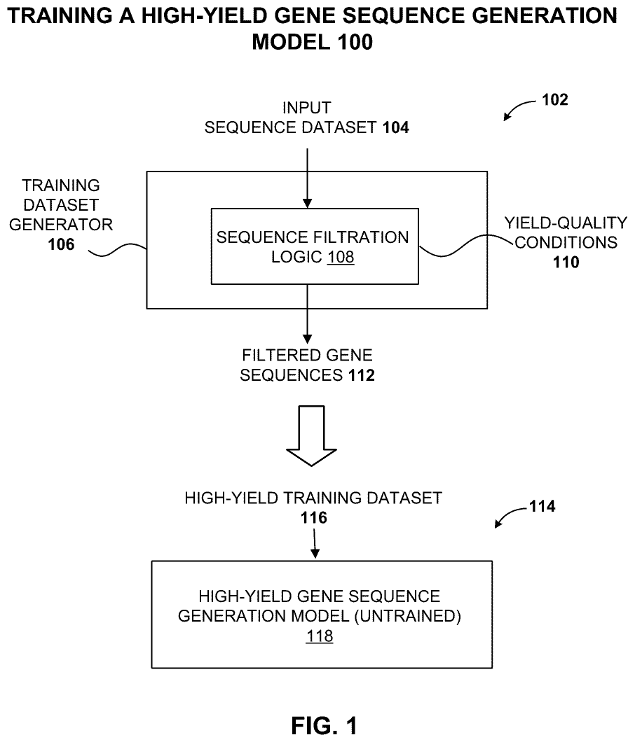

FIG. 1 is a diagram showing one example of training a high-yield gene sequence generation model 100. As shown at reference numeral 102, a sequence dataset 104 is input into training dataset generator 106 for sequence filtration to generate a high-yield training dataset. Input sequence dataset 104 includes a plurality of gene sequences to be filtered to generate the high-yield training dataset. One skilled in the art would appreciate that the number of gene sequences utilized as input sequence dataset 104 can be in any range sufficient to generate the high-yield training dataset. For example, the number of gene sequences utilized as input sequence dataset 104 can be in the range of hundreds, thousands, ten thousands, hundred thousands, or millions.

As input sequence dataset 104 is input into training dataset generator 106, sequence filtration logic 108 undergoes the process of gene sequence filtration by utilizing one or more yield-quality conditions 110. As further discussed below with respect to FIGS. 6-7, the yield-quality conditions can include an abundance condition, a stability condition, an expression condition, and/or a translation efficiency condition. In one example, only one yield quality condition 110 is utilized by training dataset generator 106. However, in other examples, two or more yield-quality conditions can be utilized by training dataset generator 106. Each yield-quality condition 110 has associated thresholds that each gene sequence of input sequence dataset 104 is compared to. Gene sequences of input sequence dataset 104 that satisfy the associated threshold(s) of at least one of the utilized yield-quality conditions 110 proceed as filtered gene sequences 112. Accordingly, filtered gene sequences 112 comprise gene sequences from input sequence dataset 104 that satisfy at least one utilized yield-quality condition 110. In one example, filtered gene sequences 112 are comprised of gene sequences that satisfy each utilized yield-quality condition 110. As shown at reference numeral 114, filtered gene sequences 112 can be utilized as a high-yield training dataset 116 to train high-yield gene sequence generation model 118.

FIG. 2 is a flowchart showing one example operation of training a high-yield gene sequence generation model. Operation 200 begins at block 210 where a plurality of gene sequence inputs are received as an input dataset. As indicated by blocks 212-220, each gene sequence within the received input dataset includes one or more metrics indicative of yield-quality that can be compared to associated thresholds for dataset filtration. The metrics can be, in one example, ground-truth metrics (e.g., characterized in a unified experimental setting). For example, as indicated by block 212, each gene sequence within the input dataset can include an abundance metric. The abundance metric, when compared to an associated threshold, provides an indication of whether a given gene sequence has high abundance (e.g., satisfies an abundance condition, described below with respect to block 232). The abundance metric can be indicative of, for example, mRNA abundance and/or protein abundance for a given gene sequence. mRNA abundance refers to the quantity of a specific mRNA molecule present in a cell at a given time. Protein abundance refers to the concentration of a specific protein in a cell.

As indicated by block 214, each gene sequence within the input dataset can also include a stability metric. The stability metric, when compared to an associated threshold, provides an indication of whether a given gene sequence has high stability (e.g., satisfies a stability condition, described below with respect to block 234). The stability metric can be indicative of, for example, mRNA stability and/or protein stability for a given gene sequence. mRNA stability refers to the lifespan of an mRNA molecule before it (the mRNA molecule) is degraded. Protein stability refers to the lifespan of a protein molecule before it (the protein molecule) is degraded.

As indicated by block 216, each gene sequence within the input dataset can also include an expression metric. The expression metric provides an indication of whether a given gene sequence has stable/consistent and/or ubiquitous expression (e.g., satisfies an expression condition, described below with respect to block 236). The expression metric can be indicative of, as noted above, stable/consistent expression and/or ubiquitous expression. Stable/consistent expression refers to genes that are expressed at stable, consistent levels across different cell types and conditions. Ubiquitous expression bears many similarities to stable/consistent expression and refers to genes that are expressed in the majority of the cells of an organism.

As indicated by block 218, each gene sequence within the input dataset can also include a translation efficiency metric. The translation efficiency metric, when compared to an associated threshold, provides an indication of whether a given gene sequence has high translation efficiency (e.g., satisfies a translation efficiency condition, described below with respect to block 234). Translation efficiency refers to a measure of how efficiently mRNA is converted into protein.

As indicated by block 220, it is expressly contemplated that the yield-quality metrics can be metric predictions rather than ground-truth metrics that have been characterized in a unified experimental setting. In one example, the yield-quality metrics can be predicted by inference via GeneCull framework regressors. However, in other examples, the yield-quality metrics can be predicted in other ways as well. Additionally, it is expressly contemplated that other the yield-quality metrics can also be utilized, as indicated by block 222.

Operation 200 proceeds at block 230, where each gene sequence of the input dataset is compared to an associated threshold. As indicated by blocks 232-240, the yield-quality metric identified for each gene sequence of the received gene input dataset can be compared to a respective threshold to determine if each gene sequence satisfies a yield-quality condition. For example, as indicated by block 232, an abundance metric of a given gene sequence can be compared to an associated threshold to determine if the given gene sequence satisfies an abundance condition. As indicated by block 234, a stability metric of a given gene sequence can be compared to an associated threshold to determine if the given gene sequence satisfies a stability condition. As indicated by block 236, an expression metric of a given gene sequence can be compared to an associated threshold to determine if the given gene sequence satisfies an expression condition. As indicated by block 238, a translation efficiency metric of a given gene sequence can be compared to an associated threshold to determine if the given gene sequence satisfies a translation efficiency condition.

In one example, as indicated above, one associated threshold can be utilized to determine if a gene sequence satisfies a respective yield-quality condition. For instance, the threshold can include an RPKM-based threshold, where an abundance metric indicative of an RPKM measurement is compared to the RPKM-based threshold. In this example, if the RPKM measurement meets or exceeds the RPKM-based threshold, the abundance condition for a given gene sequence is satisfied, indicating that the gene sequence is mRNA abundant. In another example, the threshold can include a PPT-based threshold, where a translation efficiency metric indicative of a PPT measurement is compared to the PPT-based threshold. In this example, if the PPT measurement meets or exceeds the PPT-based threshold, the translation efficiency condition for a given gene sequence is satisfied, indicating that the gene sequence efficiently converts mRNA into protein. Of course, it is expressly contemplated that other associated thresholds can be utilized as well to indicate whether an associated yield-quality condition is satisfied, as discussed in more detail below with respect to FIGS. 6-7. Moreover, it is expressly contemplated that other yield-quality conditions can also be utilized as well, as indicated by block 240.

Operation 200 proceeds at block 250 where, based on the threshold comparison, gene sequences that do not satisfy the utilized yield-quality condition are omitted from the gene sequence dataset. For example, gene sequences that do not satisfy a utilized abundance condition are omitted. In another example, gene sequences that do not satisfy a utilized stability condition are omitted. In another example gene sequences that do not satisfy a utilized expression condition are omitted. In another example, gene sequences that do not satisfy a utilized translation efficiency condition are omitted.

Operation 200 proceeds at block 260 where a deep generative model (DGM) is trained based on the filtered high-yield dataset. Because the input dataset has been filtered to omit sequences that do not satisfy a given yield-quality condition, each remaining gene sequence contains a yield-quality metric that satisfies the respective yield-quality condition and is therefore representative of a high-yield quality characteristic. For example, gene sequences that satisfy the abundance condition are indicative of having high mRNA abundance or protein abundance. In another example, gene sequences that satisfy the stability condition are indicative of having high mRNA stability or protein stability. In another example, gene sequences that satisfy the expression condition are indicative of having stable/consistent or ubiquitous expression. In another example, gene sequences that satisfy the translation efficiency condition indicate that the gene sequence efficiently converts mRNA into protein. Training a DGM on a dataset following a certain distribution (e.g., a distribution encompassing high-yield gene sequences as denoted by their yield-quality condition) supports the generation of data points following the same distribution. Thus, by utilizing the filtered gene sequence dataset to train a DGM, high-yield gene sequence predictions can be generated for subsequent dataset inputs. As indicated by block 262, a language model can be trained on the high-yield training dataset. As indicated by block 264, a variational autoencoder (VAE) can be trained on the high-yield training dataset. As indicated by block 266, a generative adversarial network (GAN) can be trained on the high-yield training dataset. As indicated by block 268, a diffusion model can be trained on the high-yield training dataset. As indicated by block 270, an energy-based model can be trained on the high-yield training dataset. As indicated by block 272, a flow-based model can be trained on the high-yield training dataset. Additionally, it is expressly contemplated that other models can be trained on the high-yield training dataset as well, as indicated by block 274.

FIG. 3 is a flowchart showing another example operation of training a high-yield gene sequence generation model. FIG. 3 bears some similarities to FIG. 2., and like components are numbered accordingly. Operation 300 begins at block 310, where a plurality of gene sequence inputs are received as an input dataset. As indicated by blocks 312-320, each gene sequence within the received input dataset includes one or more metrics indicative of yield-quality that can be compared to associated thresholds for dataset filtration. The metrics can be, in one example, ground-truth metrics (e.g., characterized in a unified experimental setting). For example, as indicated by block 312, each gene sequence within the input dataset can include an abundance metric. A given abundance metric, when compared to an associated threshold, provides an indication of whether a given gene sequence has high abundance (e.g., satisfies an abundance condition, described below with respect to block 332). The abundance metric can be indicative of, for example, mRNA abundance and/or protein abundance for a given gene sequence. mRNA abundance refers to the quantity of a specific mRNA molecule present in a cell at a given time. Protein abundance refers to the concentration of a specific protein in a cell.

As indicated by block 314, each gene sequence within the input dataset can alternatively or additionally include a stability metric. A given stability metric, when compared to an associated threshold, provides an indication of whether a given gene sequence has high stability (e.g., satisfies a stability condition, described below with respect to block 334). The stability metric can be indicative of, for example, mRNA stability and/or protein stability for a given gene sequence. mRNA stability refers to the lifespan of an mRNA molecule before it (the mRNA molecule) is degraded. Protein stability refers to the lifespan of a protein molecule before it (the protein molecule) is degraded.

As indicated by block 316, each gene sequence within the input dataset can alternatively or additionally include an expression metric. A given expression metric, when compared to an associated threshold, provides an indication of whether a given gene sequence has stable/consistent and/or ubiquitous expression (e.g., satisfies an expression condition, described below with respect to block 336). The expression metric can be indicative of, as noted above, stable/consistent expression and/or ubiquitous expression. Stable/consistent expression refers to genes that are expressed at stable, consistent levels across different cell types and conditions. Ubiquitous expression bears many similarities to stable/consistent expression and refers to genes that are expressed in the majority of the cells of an organism.

As indicated by block 318, each gene sequence within the gene input dataset can alternatively or additionally include a translation efficiency metric. A given translation efficiency metric, when compared to an associated threshold, provides an indication of whether a given gene sequence has high translation efficiency (e.g., satisfies a translation efficiency condition, described below with respect to block 334). Translation efficiency refers to a measure of how efficiently mRNA is converted into protein.

As indicated by block 320, it is expressly contemplated that each gene sequence within the input dataset can include a combination of metrics, wherein each identified metric can be compared to a respective threshold, discussed below. For example, each gene sequence can include both an abundance metric and a stability metric. In another example, each gene sequence can include both an expression metric and a translation efficiency metric. In another example, each gene sequence can include each of an abundance metric, stability metric, expression metric, and translation efficiency metric. Of course, one skilled in the art would appreciate that the above-referenced combinations are discussed only by way of example, and any metric or combination of metrics can be identified in each gene sequence of the gene sequence input dataset, as indicated by block 322.

As indicated by block 324, it is also expressly contemplated that the yield-quality metrics can be metric predictions rather than ground-truth metrics that have been characterized in a unified experimental setting. In one example, the yield-quality metrics can be predicted by inference via GeneCull framework regressors. Additionally, it is expressly contemplated that the yield-quality metrics can be predicted in other ways as well.

Operation 300 proceeds at block 330, where each gene sequence of the input dataset is compared to two or more associated thresholds. As indicated by blocks 332-340, each yield-quality metric identified for each gene sequence of the received gene sequence input dataset can be compared to a respective threshold to determine if each gene sequence satisfies a yield-quality condition. For example, as indicated by block 332, an abundance metric of a given gene sequence can be compared to associated thresholds to determine if the given gene sequence satisfies an abundance condition. As indicated by block 334, a stability metric of a given gene sequence can be compared to associated thresholds to determine if the given gene sequence satisfies a stability condition. As indicated by block 336, an expression metric of a given gene sequence can be compared to associated thresholds to determine if the given gene sequence satisfies an expression condition. As indicated by block 338, a translation efficiency metric of a given gene sequence can be compared to associated thresholds to determine if the given gene sequence satisfies a translation efficiency condition.

As indicated by block 340, a combination of metrics and/or conditions can be utilized. In particular, at block 340, it is expressly contemplated that each gene sequence within the gene sequence input dataset can be compared to associated thresholds to determine a combination of yield-quality conditions. For example, each gene sequence can be compared to associated thresholds to determine both an abundance condition and a stability condition. In another example, each gene sequence can be compared to associated thresholds to determine both an expression condition and a translation efficiency condition. In another example, each gene sequence can be compared to associated thresholds to determine each of an abundance condition, stability condition, expression condition, and translation efficiency condition. Of course, one skilled in the art would appreciate that the above-referenced combinations are discussed only by way of example, and any yield-quality condition or combination of yield-quality conditions can be utilized as well, as discussed in more detail below with respect to FIGS. 6-7. Additionally, it is expressly contemplated that other yield-quality conditions can be utilized as well, as indicated by block 342.

Operation 300 proceeds at block 350 where a consensus is formed based on comparison 330. As indicated by block 352, the input dataset can be processed to form a union consensus. As indicated by block 354, the input dataset can also be processed to form an intersectional consensus.

A union consensus can be formed at one or both of a metric level or a condition level. A union consensus at the metric level is a consensus that is formed by allowing gene sequences from the gene sequence input dataset that merge at least two utilized thresholds of a plurality of utilized thresholds for a given yield-quality to proceed, and omitting gene sequences that do not meet at least two utilized thresholds. At the metric level, the outputs from applying the union consensus on a set of sequence inputs are the sequences that merge at least two of the utilized thresholds. For example, if an abundance condition indicating mRNA abundance is utilized, a union consensus may be applied. For instance, two or more associated thresholds can be utilized to determine if a gene sequence satisfies the abundance condition. More specifically, an abundance condition can include any combination of an RPKM-based threshold, an FPKM-based threshold, and/or a TPM-based threshold, where abundance metrics indicative of an RPKM measurement, FPKM measurement, and/or TPM measurement is compared to its corresponding threshold. In this example, if at least two measurements meet or exceed their respective threshold, the abundance condition for a given gene sequence is satisfied, indicating that the gene sequence is mRNA abundant. In another example, an abundance condition can include any combination of a PPM-based threshold, a yield-based threshold, and/or a titer-based threshold, where abundance metrics indicative of a PPM measurement, yield measurement, and/or titer measurement is compared to its corresponding threshold. In this example, if at least two measurements meet or exceed their respective threshold, the abundance condition for a given gene sequence is satisfied, indicating that the gene sequence is protein abundant. Of course, it is expressly contemplated that other associated thresholds or combination of thresholds can be utilized as well for each respective yield-quality condition, as discussed in more detail below with respect to FIGS. 6-7.

An intersectional consensus can also be formed at one or both of a metric level or a condition level. An intersectional consensus at the metric level is a consensus that is formed by allowing gene sequences from the gene sequence input dataset that overlap between each utilized threshold of a plurality of utilized thresholds for a given yield-quality metric to proceed, and omitting gene sequences that do not meet each utilized threshold. At the metric level, the outputs from applying the intersectional consensus on a set of sequence inputs are the sequences that overlap between each of the utilized thresholds. From a similar example as above, if an abundance condition indicating mRNA abundance is utilized, an intersectional consensus can be applied. For instance, two or more associated thresholds can be utilized to determine if a gene sequence satisfies the abundance condition. More specifically, an abundance condition can include any combination of an RPKM-based threshold, an FPKM-based threshold, and/or a TPM-based threshold, where abundance metrics indicative of an RPKM measurement, FPKM measurement, and/or TPM measurement is compared to its corresponding threshold. In this example, if each measurement meets or exceeds its respective threshold, the abundance condition for a given gene is satisfied, indicating that the gene sequence is mRNA abundant. In another example, an abundance condition can include any combination of a PPM-based threshold, a yield-based threshold, and/or a titer-based threshold, where abundance metrics indicative of a PPM measurement, yield measurement, and/or titer measurement is compared to its corresponding threshold. In this example, each measurement meets or exceeds its respective threshold, the abundance condition for a given gene sequence is satisfied, indicating that the gene sequence is protein abundant. Of course, it is expressly contemplated that other associated thresholds or combinations of thresholds can be utilized as well for each respective yield-quality condition, as discussed in more detail below with respect to FIGS. 6-7.

A union consensus at the condition level is a consensus that is formed by combining the gene sequences for each utilized yield-quality condition from the filtered input dataset in an additive process to form a high-yield training dataset. At the condition level, the outputs from applying the union consensus on the filtered sequence inputs are sequences that merge at least two utilized yield-quality conditions. For example, if an abundance condition and a stability condition are utilized, a union consensus may be applied. In this example, the gene sequences from the filtered input dataset that satisfy the abundance condition and the gene sequences from the filtered input dataset that satisfy the stability condition are merged to form the high-yield training dataset. In another example, if an abundance condition, a stability condition, an expression condition, and a translation efficiency condition are utilized, the gene sequences from the filtered input dataset that satisfy at least two of the aforementioned yield-quality conditions are merged to form the high-yield training dataset. Of course, one skilled in the art would appreciate that the above-referenced combinations are discussed only by way of example, and any combination of yield-quality conditions can be utilized as well, as discussed in more detail below with respect to FIGS. 6-7.

An intersectional consensus at the condition level is a consensus that is formed by allowing gene sequences from the filtered input dataset that meet each utilized yield-quality condition to proceed and omitting gene sequences that do not meet each utilized yield-quality condition to form a high-yield training dataset. At the condition level, the outputs from applying the intersectional consensus on the filtered sequence inputs are sequences that overlap between each utilized yield-quality condition. For example, if an abundance condition and a stability condition are utilized, an intersectional consensus may be applied. In this example, the gene sequences from the filtered input dataset that overlap between both the abundance condition and the stability condition proceed, and the gene sequences that do not overlap between both conditions are omitted to form the high-yield training dataset. In another example, if an abundance condition, a stability condition, an expression condition, and a translation efficiency condition are utilized, the gene sequences from the filtered input dataset that overlap between each of the abundance condition, stability condition, expression condition, and translation condition proceed, and sequences that do not overlap between each condition are omitted to form the high-yield training dataset. Of course, one skilled in the art would appreciate that the above-referenced combinations are discussed only by way of example, and any combination of yield-quality conditions can be utilized as well, as discussed in more detail below with respect to FIGS. 6-7.

Operation 300 proceeds at block 360, where gene sequences from the input dataset are filtered out and omitted if they do not satisfy a utilized yield-quality condition and/or utilized consensus. For example, if an abundance condition and a union consensus are utilized, gene sequences that do not satisfy the abundance condition by meeting or exceeding at least two utilized yield-quality thresholds are omitted. In another example, if an abundance condition and an intersectional consensus are utilized, gene sequences that do not satisfy the abundance condition by meeting or exceeding each utilized yield-quality threshold are omitted.

Operation 300 proceeds at block 380 where a deep generative model (DGM) is trained based on the filtered high-yield dataset. Because the input dataset has been filtered to omit sequences that do not satisfy at least two utilized yield-quality conditions (e.g., a union consensus) or each utilized yield-quality condition (e.g., an intersectional consensus), each remaining gene sequence is representative of at least one high-yield quality characteristic. For example, gene sequences that satisfy the abundance condition are indicative of having high mRNA abundance or protein abundance. In another example, gene sequences that satisfy the stability condition are indicative of having high mRNA stability or protein stability. In another example, gene sequences that satisfy the expression condition are indicative of having stable/consistent or ubiquitous expression. In another example, gene sequences that satisfy the translation efficiency condition indicate that the gene sequence efficiently converts mRNA into protein. Training a DGM on a dataset following a certain distribution (e.g., a distribution encompassing high-yield gene sequences as denoted by their yield-quality condition) supports the generation of data points following the same distribution. Thus, by utilizing the filtered gene sequence dataset to train a DGM, high-yield gene sequence predictions can be generated for subsequent dataset inputs. As indicated by block 382, a language model can be trained on the high-yield training dataset. As indicated by block 384, a variational autoencoder (VAE) can be trained on the high-yield training dataset. As indicated by block 386, a generative adversarial network (GAN) can be trained on the high-yield training dataset. As indicated by block 388, a diffusion model can be trained on the high-yield training dataset. As indicated by block 390, an energy-based model can be trained on the high-yield training dataset. As indicated by block 392, a flow-based model can be trained on the high-yield training dataset. Additionally, it is expressly contemplated that other models can be trained on the high-yield training dataset as well, as indicated by block 394.

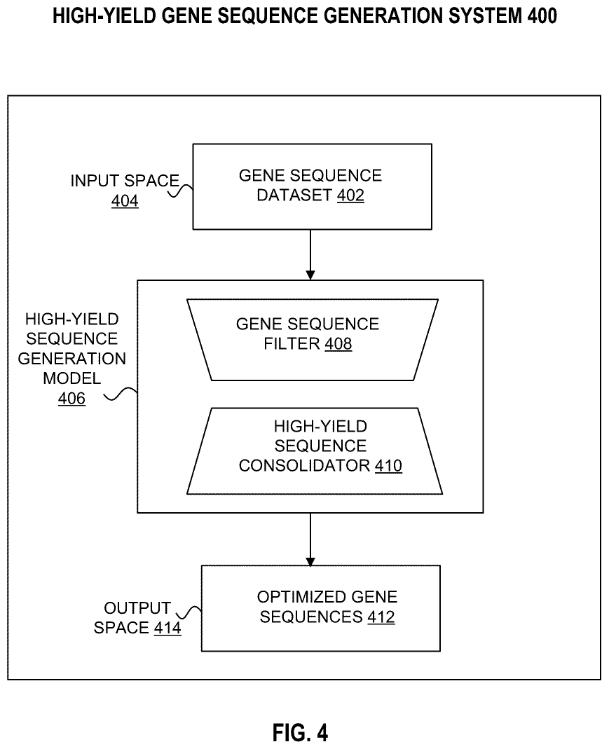

FIG. 4 is a diagram showing one example of a high-yield gene sequence generation system. System 400 illustratively includes a gene sequence dataset being received in input space 404. Gene sequence dataset 402 can be received by, for example, an external data store. Upon receiving gene sequence dataset 402 in input space 404, gene sequence dataset 402 is passed to high-yield sequence generation model 406. As shown in FIG. 4, high-yield sequence generation model 406 illustratively includes gene sequence filter 408 and optionally includes high-yield sequence consolidator 410. Gene sequence filter 408 is configured to filter gene sequences from gene sequence dataset 402 based on utilized yield-quality conditions and associated thresholds. The thresholds that can be utilized are discussed in more detail below with respect to FIGS. 6-7. Additionally, gene sequence filter 408 can filter or otherwise prune gene sequences from gene sequence dataset in the manners discussed above with respect to FIGS. 2-3. High-yield sequence consolidator 410 is configured to consolidate filtered gene sequences from gene sequence filter 408 to generate optimized gene sequences 412. In some examples, high-yield sequence consolidator 410 is configured to consolidate filtered gene sequences by forming a union consensus and/or an intersectional consensus to generate optimized gene sequences 512. The union consensus and/or intersectional consensus can be formed by, for example, utilizing the same methods described above with respect to FIG. 3. Upon consolidation of the filtered gene sequences to form the optimized gene sequences 412, the dataset including the optimized gene sequences 412 is output into output space 414. Of course, utilization of high-yield sequence consolidator 410 is optional, and it is expressly contemplated that optimized gene sequences 412 can be output to output space 414 without the utilization of high-yield sequence consolidator 410.

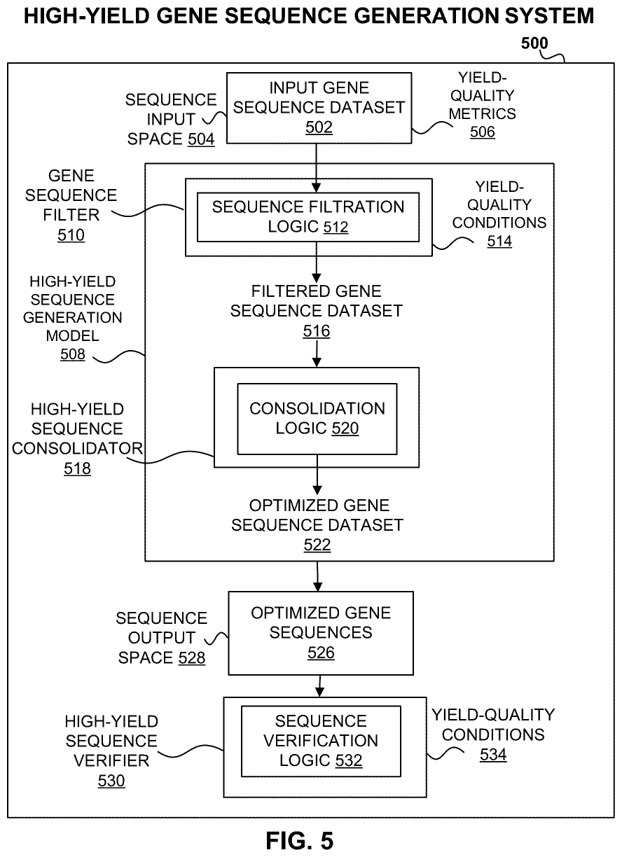

FIG. 5 is a diagram showing one example of a high-yield gene sequence generation system. System 500 illustratively includes input gene sequence dataset 502 that is received in sequence input space 504. As shown, each gene sequence of input gene sequence dataset 502 illustratively includes one or more yield-quality metrics 506. Yield-quality metrics 506 comprise one or more measurements that can be compared to associated thresholds to denote yield quality. For example, yield-quality metrics can include any measurement or combination of measurements that can be compared to associated thresholds set forth below in FIGS. 6-7. Additionally, it is expressly contemplated that other yield-quality metrics can be utilized as well.

Input gene sequence dataset 502 having one or more yield-quality metrics 506 is passed from sequence input space 504 to high-yield sequence generation model 508, and particularly to gene sequence filter 510. As shown, gene sequence filter 510 illustratively includes sequence filtration logic 512. Sequence filtration logic 512 is configured to utilize one or more yield-quality conditions 514 to generate filtered gene sequence dataset 516. The yield-quality conditions utilized by gene sequence filter 510 can include an abundance condition, stability condition, expression condition, a translation efficiency condition, or any combination thereof. In some examples, yield-quality conditions 514 comprise two or more yield-quality thresholds that are compared to respective yield-quality metrics 506. For instance, if an abundance condition is utilized, yield-quality metrics 506 including two or more of an RPKM measurement, an FPKM measurement, a TPM measurement, a PPM measurement, a yield measurement, and/or a titer measurement can be compared to a respective threshold to determine if the abundance condition is satisfied. Gene sequences that satisfy the abundance condition proceed as filtered gene sequence dataset 516, and gene sequences that fail to satisfy the abundance condition are omitted. Of course, one skilled in the art would appreciate that the use of an abundance condition is merely by way of example, and any condition and associate threshold(s) discussed below with respect to FIGS. 5-6 can be utilized as well by gene sequence filter 510.

After filtering input gene sequence dataset 502 to generate filtered gene sequence dataset 516, filtered gene sequence dataset 516 is passed to high-yield sequence consolidator 518. High-yield sequence consolidator 518 is configured to consolidate filtered gene sequence dataset 516 to generate high-yield gene sequence dataset 522. In some examples, high-yield sequence consolidator 518 is configured to consolidate filtered gene sequences by forming a union consensus and/or an intersectional consensus to generate the optimized gene sequence dataset 522. The union consensus and/or intersectional consensus can be formed by, for example, utilizing the same methods described above with respect to FIG. 3.

Upon consolidating filtered gene sequence dataset 516 to produce optimized gene sequence dataset 522, optimized gene sequences 526 are output into sequence output space 532. After generating and outputting optimized gene sequences 526, optimized gene sequences 526 can optionally be filtered again by high-yield sequence verifier 530 having sequence verification logic 532. As shown, sequence verification logic 532 utilizes yield-quality conditions 534 to re-filter and otherwise verify optimized gene sequences 526. In one example, the yield-quality conditions 534 utilized by high-yield sequence verifier 530 are the same yield-quality conditions as yield-quality conditions 514 utilized by gene sequence filter 510. However, in other examples, yield-quality conditions 534 can be different yield-quality conditions than yield-quality conditions 514. Thus, optimized gene sequences 526 can be filtered again to omit any gene sequence that does not satisfy the one or more yield-quality conditions 526. Of course, sequence verification and/or re-filtering by high-yield sequence verifier 530 is optional, and it is expressly contemplated that optimized gene sequences 526 can be output to sequence output space 528 and not undergo subsequent verification and/or re-filtering by high-yield sequence verifier 530.

FIG. 6 is a diagram showing one example generation of a high-yield training dataset 600. The embodiment shown in FIG. 6 portrays in detail the operation of filtering gene sequence inputs according to one or more yield-quality conditions. The operation can occur by, for example, any of the processes and/or systems discussed above with respect to FIGS. 1-5. As shown, a plurality of gene sequences from sequence database 602 are utilized as an input and compared to associated threshold(s) of one or more gene sequence conditions 610. Gene sequence conditions 610 can include, for example, a stability condition 612, an abundance condition 614, an expression condition 616, and/or a translation efficiency condition 618. In one example, only one gene sequence condition 610 is utilized. However, in other examples, any combination of gene sequence conditions 610 can be utilized.

As shown at reference numeral 620, stability condition 612 can indicate which gene sequences have mRNA stability and/or protein stability. For example, gene sequences input by sequence database 602 can be compared to one or more associated thresholds 624. For instance, the input gene sequences can be compared to at least one of an mRNA half-life-based threshold and/or a degradation rates-based threshold to indicate mRNA stability.

If a given gene sequence meets or exceeds at least one of the mRNA half-life-based threshold and/or the degradation-rates-based threshold, the stability condition is regarded as satisfied, indicating that the given gene sequence has mRNA stability. Further, if a given gene sequence does not meet at least one of the mRNA half-life-based threshold and/or the degradation-rates-based threshold, the stability condition is regarded as not satisfied, indicating that the given gene sequence does not have mRNA stability and should be omitted.

In another example, the input gene sequences can be compared to at least one of a protein half-life-based threshold and/or a degradation rates-based threshold to indicate protein stability. If a given gene sequence meets or exceeds at least one of the protein half-life-based threshold and/or the degradation-rates-based threshold, the stability condition is regarded as satisfied, indicating that the given gene sequence has protein stability. Further, if a given gene sequence does not meet at least one of the protein half-life-based threshold and/or the degradation-rates-based threshold, the stability condition is regarded as not satisfied, indicating that the given gene sequence does not have protein stability and should be omitted.

In one example, only one associated threshold 624 can be utilized to determine if a given gene sequence satisfies stability condition 612. However, in another example, a union consensus can be formed at the metric level for stability condition 612, in which each gene sequence input from sequence database 602 is compared to at least two associated thresholds 624. In this example, if a given gene sequence satisfies at least two of the utilized associated thresholds 624, the gene sequence is regarded as satisfying the stability condition. By way of example, if an mRNA half-life-based threshold and a degradation-rates-based threshold are utilized, each input gene sequence may be compared to both the mRNA half-life-based threshold and the degradation-rates-based threshold. If a given gene sequence meets or exceeds at least two of the mRNA half-life-based threshold or the degradation-rates-based threshold, the given gene sequence is regarded as satisfying the stability condition. Of course, the utilization of the mRNA half-life-based threshold and the degradation-rates-based threshold are only for the purposes of example, and it is expressly contemplated that any combination of associated thresholds 624 can be utilized to form a union consensus at the metric level. For example, an mRNA half-life-based threshold and a protein half-life-based threshold can be utilized. In another example, a protein half-life-based threshold and a degradation rates-based threshold can be utilized. In another example, any other combination of associated thresholds 624 can be utilized as well.

As shown at reference numeral 626, abundance condition 614 can indicate which gene sequences have mRNA abundance and/or protein abundance. For example, gene sequences input by sequence database 602 can be compared to one or more associated thresholds 628. For instance, the input gene sequences can be compared to at least one of an RPKM-based threshold, FPKM-based threshold, and/or a TPM-based threshold to indicate mRNA abundance. If a given gene sequence meets or exceeds at least one of the RPKM-based threshold, FPKM-based threshold, and/or the TPM-based threshold, the abundance condition is regarded as satisfied, indicating that the given gene sequence has mRNA abundance. Further, if a given gene sequence does not meet at least one of the RPKM-based threshold, FPKM-based threshold, and/or the TPM-based threshold, the abundance condition is regarded as not satisfied, indicating that the given gene sequence does not have mRNA abundance and should be omitted.

In another example, the input gene sequences can be compared to at least one of a PPM-based threshold, yield-based threshold, and/or a titer-based threshold to indicate protein abundance. If a given gene sequence meets or exceeds at least one of the PPM-based threshold, yield-based threshold, and/or titer-based threshold, the abundance condition is regarded as satisfied, indicating that the given gene sequence has protein abundance. Further, if a given gene sequence does not meet at least one of the PPM-based threshold, yield-based threshold, and/or titer-based threshold, the abundance condition is regarded as not satisfied, indicating that the given gene sequence does not have protein abundance and should be omitted.

In one example, only one associated threshold 628 can be utilized to determine if a given gene sequence satisfies abundance condition 614. However, in another example, a union consensus can be formed at the metric level for abundance condition 614, in which each gene sequence input from sequence database 602 is compared to at least two associated threshold 628. In this example, if a given gene sequence satisfies at least two of the utilized associated thresholds 628, the gene sequence is regarded as satisfying the abundance condition. By way of example, if an RPKM-based threshold, an FPKM-based threshold, and a TPM-based threshold are utilized, each input gene sequence may be compared to each of the RPKM-based threshold, FPKM-based threshold, and TPM-based threshold. If a given gene sequence meets or exceeds at least two of the RPKM-based threshold, FPKM-based threshold, or TPM-based threshold, the given gene sequence is regarded as satisfying the abundance condition. Of course, the utilization of the RPKM-based threshold, FPKM-based threshold, and TPM-based threshold are only for the purposes of example, and it is expressly contemplated that any combination of associated thresholds 628 can be utilized to form a union consensus at the metric level. For example, an RPKM-based threshold and a TPM-based threshold can be utilized. In another example, an FPKM-based threshold and a PPM-based threshold can be utilized. In another example, a true yield-based threshold and a titer-based threshold can be utilized. In another example, any other combination of associated thresholds 628 can be utilized as well.

As shown at reference numeral 630, expression condition 616 can indicate which gene sequences have stable/consistent expression and/or ubiquitous expression. For example, gene sequences input by sequence database 602 can be compared to one or more associated thresholds 634. For instance, the input gene sequences can be compared to a housekeeping genes-based threshold to indicate stable/consistent expression. If a given gene sequence meets or exceeds the housekeeping genes-based threshold, the expression condition is regarded as satisfied, indicating that the given gene sequence has stable/consistent expression. Further, if a given gene sequence does not meet the housekeeping genes-based threshold, the expression condition is regarded as not satisfied, indicating that the given gene sequence does not have stable/consistent expression and should be omitted.

In another example, the input gene sequences can be compared to a collagen-based threshold to indicate ubiquitous expression. If a given gene sequence meets or exceeds the collagen-based threshold, the expression condition is regarded as satisfied, indicating that the given gene sequence has ubiquitous expression. Further, if a given gene sequence does not meet the collagen-based threshold, the expression condition is regarded as not satisfied, indicating that the given gene sequence does not have ubiquitous expression and should be omitted.

In one example, only one associated threshold 634 can be utilized to determine if a given gene sequence satisfies expression condition 616. However, in another example, a union consensus can be formed at the metric level for expression condition 616, in which each gene sequence input from sequence database 602 is compared to at least two associated threshold 634. In this example, if a given gene sequence satisfies at least two of the utilized associated thresholds 634, the gene sequence is regarded as satisfying the expression condition. By way of example, if a housekeeping genes-based threshold and a collagen-based threshold are utilized, each input gene sequence may be compared to each of the housekeeping genes-based threshold and the collagen-based threshold. If a given gene sequence meets or exceeds at least two of the housekeeping genes-based threshold or the collagen-based threshold, the given gene sequence is regarded as satisfying the expression condition. Of course, the utilization of the housekeeping genes-based threshold and the collagen-based threshold are only for the purposes of example.

As shown at reference numeral 636, translation efficiency condition 618 can indicate which gene sequences have mRNA translation efficiency. For example, gene sequences input by sequence database 602 can be compared to one or more associated thresholds 638. For instance, the input gene sequences can be compared to at least one of a ribosomal profiling-based threshold, a PPT-based threshold, and/or a PTR-based threshold to indicate mRNA translation efficiency. If a given gene sequence meets or exceeds at least one of the ribosomal profiling-based threshold, PPT-based threshold, and/or PTR-based threshold, the translation efficiency condition is regarded as satisfied, indicating that the given gene sequence has mRNA translation efficiency. Further, if a given gene sequence does not meet at least one of the ribosomal profiling-based threshold, PPT-based threshold, and/or PTR-based threshold, the translation efficiency condition is regarded as not satisfied, indicating that the given gene sequence does not have translation efficiency and should be omitted.

In one example, only one associated threshold 638 can be utilized to determine if a given gene sequence satisfies translation efficiency condition 618. However, in another example, a union consensus can be formed at the metric level for translation efficiency condition 618, in which each gene sequence input from sequence database 602 is compared to at least two associated threshold 638. In this example, if a given gene sequence satisfies at least two of the utilized associated thresholds 638, the gene sequence is regarded as satisfying the translation efficiency condition. By way of example, if a ribosomal profiling-based threshold, a PPT-based threshold, and a PTR-based threshold are utilized, each input gene sequence may be compared to each of the ribosomal profiling-based threshold, PPT-based threshold, and PTR-based threshold. If a given gene sequence meets or exceeds at least two of the ribosomal profiling-based threshold, PPT-based threshold, or PTR-based threshold, the given gene sequence is regarded as satisfying the translation efficiency condition. Of course, the utilization of the ribosomal profiling-based threshold, PPT-based threshold, and PTR-based threshold are only for the purposes of example, and it is expressly contemplated that any combination of associated thresholds 638 can be utilized to form a union consensus at the metric level. For example, a ribosomal profiling-based threshold and a PPT-based threshold can be utilized. In another example, a ribosomal profiling-based threshold and a PTR-based threshold can be utilized. In another example, a PPT-based threshold and a PTR-based threshold can be utilized. In another example, any other combination of associated thresholds 638 can be utilized as well.

Gene sequence inputs that satisfy at least one of the utilized gene sequence conditions 610 proceed to filtered dataset consolidation/consensus step 640. In the example where only one gene sequence condition is utilized, gene sequences that satisfy the given condition proceed to filtered dataset consolidation/consensus step 640 where each gene sequence that satisfies the given condition is consolidated to form a high-yield training dataset. For example, gene sequences that satisfy stability condition 612 are consolidated as stability dataset 642. In another example, gene sequences that satisfy abundance condition 614 are consolidated at abundance dataset 644. In another example, gene sequences that satisfy expression condition 616 are consolidated at expression dataset 646. In another example, gene sequences that satisfy translation efficiency condition 618 are consolidated at efficiency dataset 648. Additionally, in an example where a union consensus was formed for a given condition 610, gene sequences that meet or exceed at least two associated thresholds for the given condition are consolidated at filtered dataset consolidation/consensus step 640.

In another example where two or more gene sequence conditions 610 are utilized, gene sequences that satisfy at least two utilized conditions proceed to filtered dataset consolidation/consensus step 640, where each gene sequence that satisfies at least two utilized conditions is consolidated to form a high-yield training dataset. In particular, a union consensus can be formed again at the condition level, where each gene sequence that satisfies at least two utilized gene sequence conditions is consolidated to form the high-yield training dataset. For example, gene sequences that satisfy a utilized abundance condition and a utilized stability condition can be consolidated. In another example, gene sequences that satisfy a utilized expression condition and a utilized efficiency condition can be consolidated. In another example, gene sequences that satisfy a utilized abundance condition, stability condition, expression condition, and/or translation efficiency condition can be consolidated. Of course, it is expressly contemplated that gene sequences that satisfy any combination of gene sequence conditions can be consolidated as well. After the gene sequences input from sequence database 602 are filtered and consolidated, the resulting high-yield training dataset is output at reference numeral 650.

FIG. 7 is a diagram showing another example generation of a high-yield training dataset. FIG. 7 bears many similarities to FIG. 6, and like components are numbered accordingly. The embodiment shown in FIG. 7 portrays in detail the operation of filtering gene sequence inputs according to a plurality of yield-quality conditions. The operation can occur by, for example, any of the processes and/or systems discussed above with respect to FIGS. 1-5. As shown, a plurality of gene sequences from sequence database 702 are utilized as an input and compared to associated threshold(s) of two or more gene sequence conditions 710. Gene sequence conditions 710 can include, for example, a stability condition 712, an abundance condition 714, an expression condition 716, and/or a translation efficiency condition 718. However, it is expressly contemplated that any combination of gene sequence conditions 710 can be utilized for high-yield training dataset generation.

As shown at reference numeral 720, stability condition 712 can indicate which gene sequences have mRNA stability and/or protein stability. For example, gene sequences input by sequence database 702 can be compared to one or more associated thresholds 724. For instance, the input gene sequences can be compared to at least one of an mRNA half-life-based threshold and/or a degradation rates-based threshold to indicate mRNA stability. If a given gene sequence meets or exceeds at least one of the mRNA half-life-based threshold and/or the degradation-rates-based threshold, the stability condition is regarded as satisfied, indicating that the given gene sequence has mRNA stability. Further, if a given gene sequence does not meet at least one of the mRNA half-life-based threshold and/or the degradation-rates-based threshold, the stability condition is regarded as not satisfied, indicating that the given gene sequence does not have mRNA stability and should be omitted.

In another example, the input gene sequences can be compared to at least one of a protein half-life-based threshold and/or a degradation rates-based threshold to indicate protein stability. If a given gene sequence meets or exceeds at least one of the protein half-life-based threshold and/or the degradation-rates-based threshold, the stability condition is regarded as satisfied, indicating that the given gene sequence has protein stability. Further, if a given gene sequence does not meet at least one of the protein half-life-based threshold and/or the degradation-rates-based threshold, the stability condition is regarded as not satisfied, indicating that the given gene sequence does not have protein stability and should be omitted.

In one example, an intersectional consensus can be formed at the metric level for stability condition 712, in which each gene sequence input from sequence database 702 is compared to at least two associated thresholds 724. In this example, if a given gene sequence satisfies each of the utilized associated thresholds 724, the gene sequence is regarded as satisfying the stability condition. By way of example, if an mRNA half-life-based threshold and a degradation-rates-based threshold are utilized, each input gene sequence may be compared to both the mRNA half-life-based threshold and the degradation-rates-based threshold. If a given gene sequence meets or exceeds each of the mRNA half-life-based threshold and the degradation-rates-based threshold, the given gene sequence is regarded as satisfying the stability condition. Of course, the utilization of the mRNA half-life-based threshold and the degradation-rates-based threshold is only for the purposes of example, and it is expressly contemplated that any combination of associated thresholds 724 can be utilized to form an intersectional consensus at the metric level. For example, an mRNA half-life-based threshold and a protein half-life-based threshold can be utilized. In another example, a protein half-life-based threshold and a degradation rates-based threshold can be utilized. In another example, any other combination of associated thresholds 724 can be utilized as well.

As shown at reference numeral 726, abundance condition 714 can indicate which gene sequences have mRNA abundance and/or protein abundance. For example, gene sequences input by sequence database 702 can be compared to one or more associated thresholds 728. For instance, the input gene sequences can be compared to at least one of an RPKM-based threshold, FPKM-based threshold, and/or a TPM-based threshold to indicate mRNA abundance. If a given gene sequence meets or exceeds at least one of the RPKM-based threshold, FPKM-based threshold, and/or the TPM-based threshold, the abundance condition is regarded as satisfied, indicating that the given gene sequence has mRNA abundance. Further, if a given gene sequence does not meet at least one of the RPKM-based threshold, FPKM-based threshold, and/or the TPM-based threshold, the abundance condition is regarded as not satisfied, indicating that the given gene sequence does not have mRNA abundance and should be omitted.

In another example, the input gene sequences can be compared to at least one of a PPM-based threshold, yield-based threshold, and/or a titer-based threshold to indicate protein abundance. If a given gene sequence meets or exceeds at least one of the PPM-based threshold, yield-based threshold, and/or titer-based threshold, the abundance condition is regarded as satisfied, indicating that the given gene sequence has protein abundance. Further, if a given gene sequence does not meet at least one of the PPM-based threshold, yield-based threshold, and/or titer-based threshold, the abundance condition is regarded as not satisfied, indicating that the given gene sequence does not have protein abundance and should be omitted.

In one example, an intersectional consensus can be formed at the metric level for abundance condition 714, in which each gene sequence input from sequence database 702 is compared to at least two associated threshold 728. In this example, if a given gene sequence satisfies each of the utilized associated thresholds 728, the gene sequence is regarded as satisfying the abundance condition. By way of example, if an RPKM-based threshold, an FPKM-based threshold, and a TPM-based threshold are utilized, each input gene sequence may be compared to each of the RPKM-based threshold, FPKM-based threshold, and TPM-based threshold. If a given gene sequence meets or exceeds each of the RPKM-based threshold, FPKM-based threshold, or TPM-based threshold, the given gene sequence is regarded as satisfying the abundance condition. Of course, the utilization of the RPKM-based threshold, FPKM-based threshold, and TPM-based threshold is only for the purpose of example, and it is expressly contemplated that any combination of associated thresholds 728 can be utilized to form an intersectional consensus at the metric level. For example, an RPKM-based threshold and a TPM-based threshold can be utilized. In another example, an FPKM-based threshold and a PPM-based threshold can be utilized. In another example, a yield-based threshold and a titer-based threshold can be utilized. In another example, any other combination of associated thresholds 728 can be utilized as well.

As shown at reference numeral 730, expression condition 716 can indicate which gene sequences have stable/consistent expression and/or ubiquitous expression. For example, gene sequences input by sequence database 702 can be compared to one or more associated thresholds 734. For instance, the input gene sequences can be compared to a housekeeping genes-based threshold to indicate stable/consistent expression. If a given gene sequence meets or exceeds the housekeeping genes-based threshold, the expression condition is regarded as satisfied, indicating that the given gene sequence has stable/consistent expression. Further, if a given gene sequence does not meet the housekeeping genes-based threshold, the expression condition is regarded as not satisfied, indicating that the given gene sequence does not have stable/consistent expression and should be omitted.

In another example, the input gene sequences can be compared to a collagen-based threshold to indicate ubiquitous expression. If a given gene sequence meets or exceeds the collagen-based threshold, the expression condition is regarded as satisfied, indicating that the given gene sequence has ubiquitous expression. Further, if a given gene sequence does not meet the collagen-based threshold, the expression condition is regarded as not satisfied, indicating that the given gene sequence does not have ubiquitous expression and should be omitted.

In one example, an intersectional consensus can be formed at the metric level for expression condition 716, in which each gene sequence input from sequence database 602 is compared to at least two associated threshold 734. In this example, if a given gene sequence satisfies each of the utilized associated thresholds 734, the gene sequence is regarded as satisfying the expression condition. By way of example, if a housekeeping genes-based threshold and a collagen-based threshold are utilized, each input gene sequence may be compared to each of the housekeeping genes-based threshold and the collagen-based threshold. If a given gene sequence meets or exceeds each of the housekeeping genes-based threshold or the collagen-based threshold, the given gene sequence is regarded as satisfying the expression condition. Of course, the utilization of the housekeeping genes-based threshold and the collagen-based threshold is only for the purposes of example.

As shown at reference numeral 736, translation efficiency condition 718 can indicate which gene sequences have mRNA translation efficiency. For example, gene sequences input by sequence database 702 can be compared to one or more associated thresholds 738. For instance, the input gene sequences can be compared to at least one of a ribosomal profiling-based threshold, a PPT-based threshold, and/or a PTR-based threshold to indicate mRNA translation efficiency. If a given gene sequence meets or exceeds at least one of the ribosomal profiling-based threshold, PPT-based threshold, and/or PTR-based threshold, the translation efficiency condition is regarded as satisfied, indicating that the given gene sequence has mRNA translation efficiency. Further, if a given gene sequence does not meet at least one of the ribosomal profiling-based threshold, PPT-based threshold, and/or PTR-based threshold, the translation efficiency condition is regarded as not satisfied, indicating that the given gene sequence does not have translation efficiency and should be omitted.

In one example, an intersectional consensus can be formed at the metric level for translation efficiency condition 718, in which each gene sequence input from sequence database 702 is compared to at least two associated thresholds 738. In this example, if a given gene sequence satisfies each of the utilized associated thresholds 738, the gene sequence is regarded as satisfying the translation efficiency condition. By way of example, if a ribosomal profiling-based threshold, a PPT-based threshold, and a PTR-based threshold are utilized, each input gene sequence may be compared to each of the ribosomal profiling-based threshold, PPT-based threshold, and PTR-based threshold. If a given gene sequence meets or exceeds each of the ribosomal profiling-based threshold, PPT-based threshold, or

PTR-based threshold, the given gene sequence is regarded as satisfying the translation efficiency condition. Of course, the utilization of the ribosomal profiling-based threshold, PPT-based threshold, and PTR-based threshold is only for the purposes of example, and it is expressly contemplated that any combination of associated thresholds 738 can be utilized to form an intersectional consensus at the metric level. For example, a ribosomal profiling-based threshold and a PPT-based threshold can be utilized. In another example, a ribosomal profiling-based threshold and a PTR-based threshold can be utilized. In another example, a PPT-based threshold and a PTR-based threshold can be utilized. In another example, any other combination of associated thresholds 738 can be utilized as well.

Gene sequence inputs that satisfy at least one of the utilized gene sequence conditions 710 proceed to filtered dataset consensus step 740, where each gene sequence that satisfies at least one utilized gene sequence condition is consolidated to form a high-yield training dataset. In particular, an intersectional consensus can be formed again at the condition level, as indicated generally by arrows 750, where each gene sequence that satisfies each utilized gene sequence condition can be consolidated to form the high-yield training dataset. For example, gene sequences that satisfy each of a utilized abundance condition and a utilized stability condition can be consolidated. In another example, gene sequences that satisfy each of a utilized expression condition and a utilized translation efficiency condition can be consolidated. In another example, gene sequences that satisfy each of a utilized abundance condition, stability condition, expression condition, and a translation efficiency condition can be consolidated. Of course, it is expressly contemplated that the gene sequences that satisfy each of any combination of gene sequence conditions can be consolidated as well. After the gene sequences input from sequence database 702 are filtered and consolidated, the resulting high-yield training dataset is output at reference numeral 760.

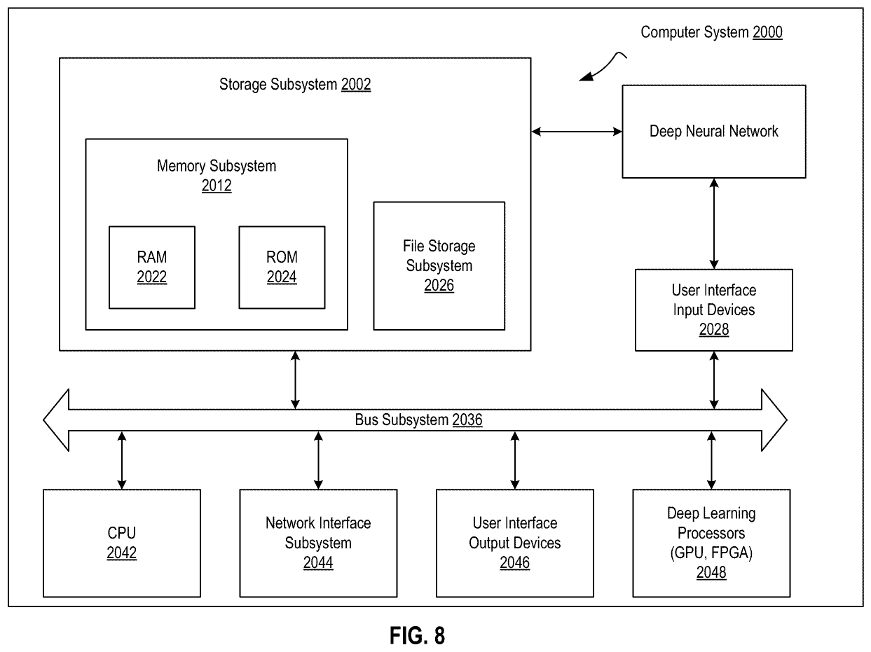

FIG. 8 shows an example computer system 2000 that can be used to implement the technology disclosed. Computer system 2000 includes at least one central processing unit (CPU) 2042 that communicates with a number of peripheral devices via bus subsystem 2026. These peripheral devices can include a storage subsystem 2002 including, for example, memory devices and a file storage subsystem 2026, user interface input devices 2028, user interface output devices 2046, and a network interface subsystem 2044. The input and output devices allow user interaction with computer system 2000. Network interface subsystem 2044 provides an interface to outside networks, including an interface to corresponding interface devices in other computer systems.

In one implementation, the deep neural network like the large language models disclosed here is communicably linked to the storage subsystem 2002 and the user interface input devices 2028.

User interface input devices 2028 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 2000.

User interface output devices 2046 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 2000 to the user or to another machine or computer system.

Storage subsystem 2002 stores programming and data constructs that provide the functionality of some or all of the modules and methods described herein. These software modules are generally executed by processors 2048.

Processors 2048 can be graphics processing units (GPUs), field-programmable gate arrays (FPGAs), application-specific integrated circuits (ASICs), and/or coarse-grained reconfigurable architectures (CGRAs). Processors 2048 can be hosted by a deep learning cloud platform such as Google Cloud Platform™, Xilinx™, and Cirrascale™. Examples of processors 2048 include Google's Tensor Processing Unit (TPU)™, rackmount solutions like GX4 Rackmount Series™, GX20 Rackmount Series™, NVIDIA DGX-1™, Microsoft' Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 2012 used in the storage subsystem 2002 can include a number of memories including a main random access memory (RAM) 2022 for storage of instructions and data during program execution and a read only memory (ROM) 2024 in which fixed instructions are stored. A file storage subsystem 2026 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 2026 in the storage subsystem 2002, or in other machines accessible by the processor.

Bus subsystem 2036 provides a mechanism for letting the various components and subsystems of computer system 2000 communicate with each other as intended. Although bus subsystem 2036 is shown schematically as a single bus, alternative implementations of the bus subsystem can use multiple busses.

Computer system 2000 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 2000 depicted in FIG. 8 is intended only as a specific example for purposes of illustrating the preferred implementations of the present invention. Many other configurations of computer system 2000 are possible having more or less components than the computer system depicted in FIG. 8.

In various implementations, a learning system is provided. In some implementations, a feature vector is provided to a learning system. Based on the input features, the learning system generates one or more outputs. In some implementations, the output of the learning system is a feature vector. In some implementations, the learning system comprises an SVM. In other implementations, the learning system comprises an artificial neural network. In some implementations, the learning system is pre-trained using training data. In some implementations training data is retrospective data. In some implementations, the retrospective data is stored in a data store. In some implementations, the learning system may be additionally trained through manual curation of previously generated outputs.

In some implementations, the sequence generator 172 is a trained classifier. In some implementations, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

FIG. 8 is a schematic of an exemplary computing node. Computing node 2000 is only one example of a suitable computing node and is not intended to suggest any limitation as to the scope of use or functionality of embodiments described herein. Regardless, computing node 2000 is capable of being implemented and/or performing any of the functionality set forth hereinabove.

In computing node 2000 there is a computer system/server, which is operational with numerous other general purpose or special purpose computing system environments or configurations. Examples of well-known computing systems, environments, and/or configurations that may be suitable for use with computer system/server include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed computing environments that include any of the above systems or devices, and the like.

Computer system/server may be described in the general context of computer system-executable instructions, such as program modules, being executed by a computer system. Generally, program modules may include routines, programs, objects, components, logic, data structures, and so on that perform particular tasks or implement particular abstract data types. Computer system/server may be practiced in distributed computing environments where tasks are performed by remote processing devices that are linked through a communications network. In a distributed computing environment, program modules may be located in both local and remote computer system storage media including memory storage devices.

As shown in FIG. 8, computer system/server in computing node 2000 is shown in the form of a general-purpose computing device. The components of computer system/server may include, but are not limited to, one or more processors or processing units, a system memory, and a bus that couples various system components including system memory to processor.

The Bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server typically includes a variety of computer system readable media. Such media may be any available media that is accessible by computer system/server, and it includes both volatile and non-volatile media, removable and non-removable media.

System memory can include computer system readable media in the form of volatile memory, such as random access memory (RAM) and/or cache memory. Algorithm

Computer system/server may further include other removable/non-removable, volatile/non-volatile computer system storage media. By way of example only, storage system can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus by one or more data media interfaces. As will be further depicted and described below, memory may include at least one program product having a set (e.g., at least one) of program modules that are configured to carry out the functions of embodiments of the disclosure.

Program/utility, having a set (at least one) of program modules, may be stored in memory by way of example, and not limitation, as well as an operating system, one or more application programs, other program modules, and program data. Each of the operating system, one or more application programs, other program modules, and program data or some combination thereof, may include an implementation of a networking environment. Program modules generally carry out the functions and/or methodologies of embodiments as described herein.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to implementations of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the FIGS. illustrate the architecture, functionality, and operation of possible implementations of systems, methods, and computer program products according to various implementations of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the FIGS. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### Performance Results as Objective Indicia of Inventiveness and Non-Obviousness

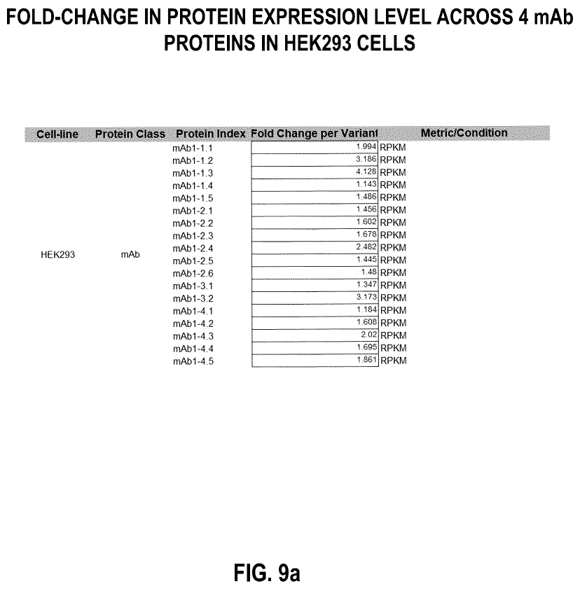

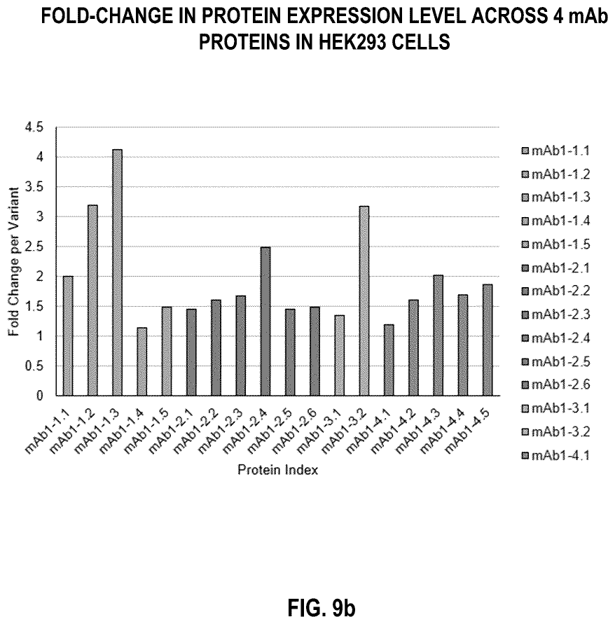

FIGS. 9A-9B include a table and a chart that show the fold-change in yield/expression for four proteins that were each expressed in the HEK293 cell line. As shown, FIG. 9A includes an identification of the metric/condition utilized for each respective protein variant where applicable. For example, as shown in FIG. 9A, an RPKM metric is utilized for each variant. Additionally, as shown in FIGS. 9A-9B, each of the four proteins has at least one variant with a 1.5-to 2-fold or more increase in protein expression relative to the wild type/reference sequence, demonstrating the successful performance of the high-yield gene sequence prediction system. In fact, two of the proteins had variants with at least a 3- to 4-fold increase in protein expression.

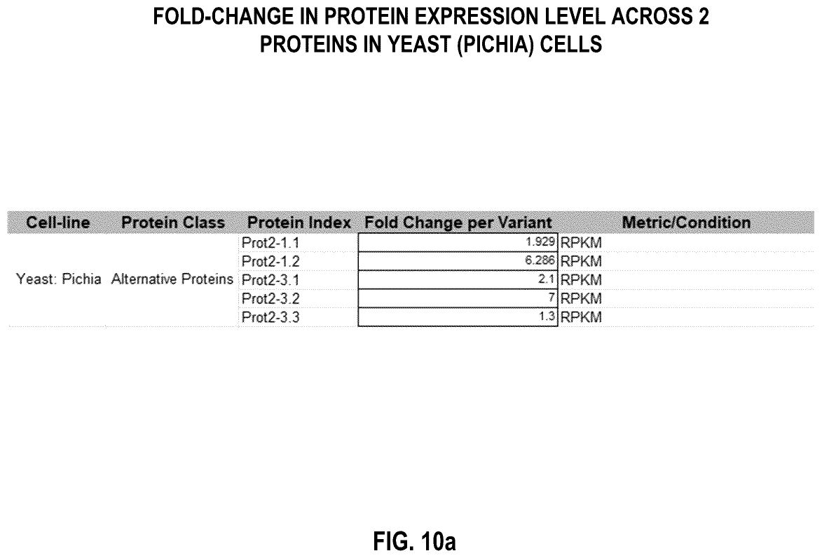

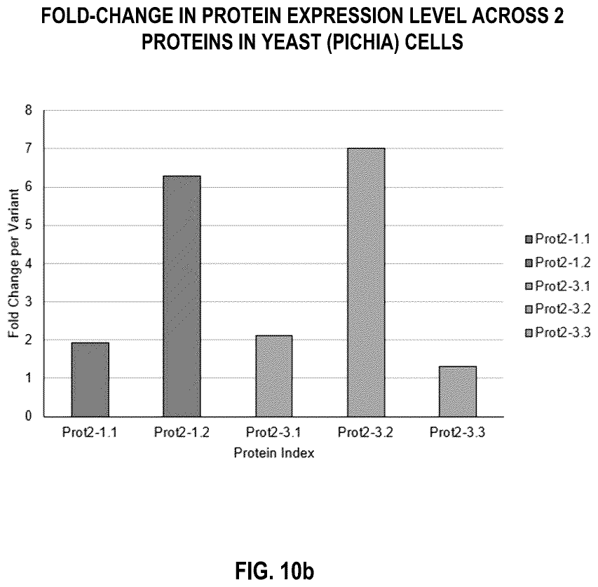

FIGS. 10A-10B include a table and a chart that show the fold-change in yield/expression for two proteins that were each expressed in the Yeast Pichia cell line. As shown, FIG. 10A includes an identification of the metric/condition utilized for each respective protein variant where applicable. For example, as shown in FIG. 10A, an RPKM metric is utilized for each variant. Additionally, As shown in FIGS. 10A-10B, the two proteins have at least one variant with a 1.5-to 2-fold or more increase in protein expression relative to the wild type/reference sequence, demonstrating the successful performance of the high-yield gene sequence prediction system. In fact, the two proteins also had variants with at least a 6- to 7-fold increase in protein expression.

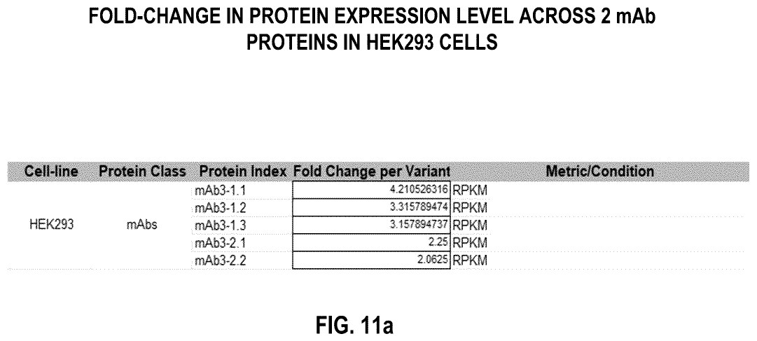

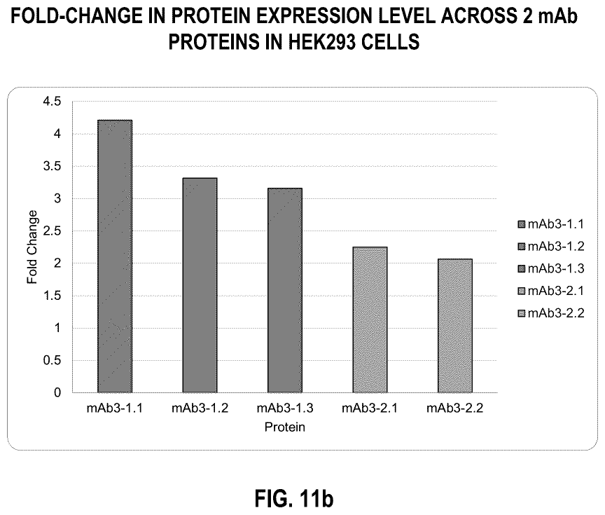

FIGS. 11A-11B include a table and a chart that shows the fold-change in yield/expression for two proteins that were each expressed in the HEK293 cell line. As shown, FIG. 11A includes an identification of the metric/condition utilized for each respective protein variant where applicable. For example, as shown in FIG. 11A, an RPKM metric is utilized for each variant. Additionally, As shown in FIGS. 11A-11B, both proteins have at least two variants with a 2-fold or more increase in protein expression relative to the wild type/reference sequence, demonstrating the successful performance of the high-yield gene sequence prediction system. In fact, one protein had each of their variants exhibit at least a 3- to 4-fold increase in protein expression.

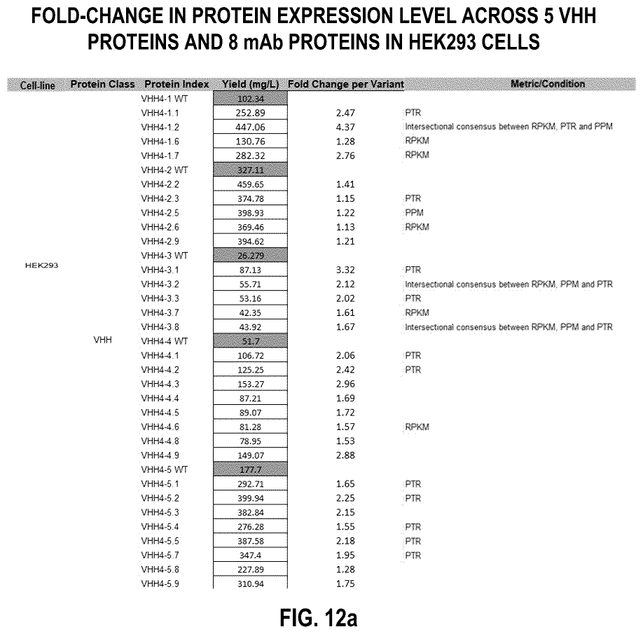

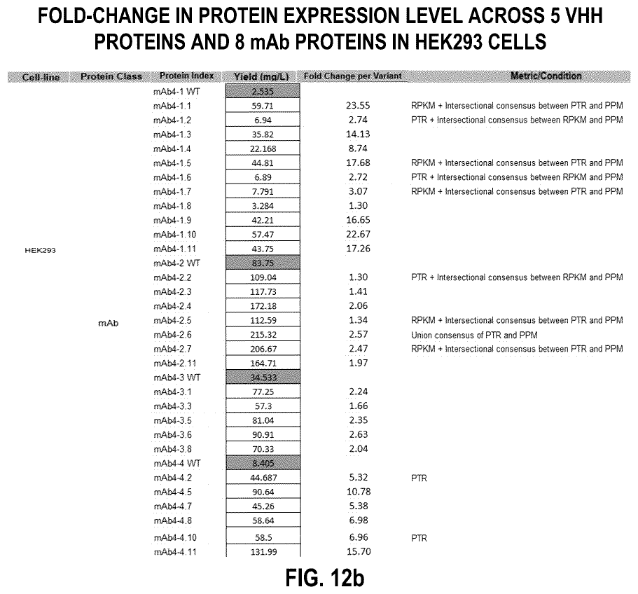

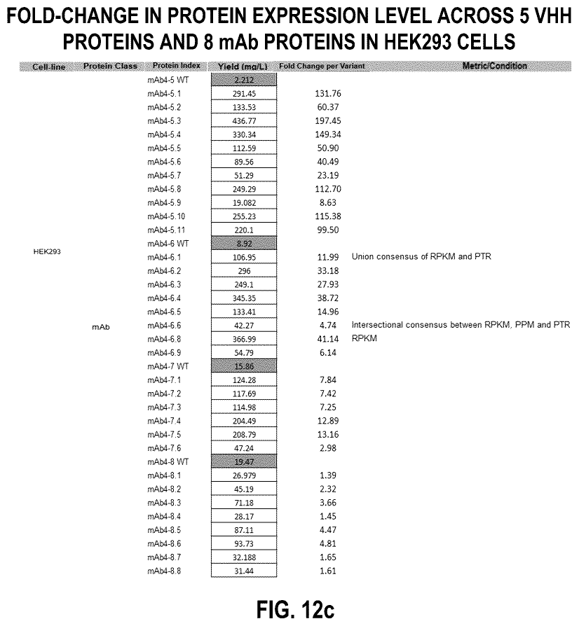

FIGS. 12A-12C includes a table that shows the fold-change in yield/expression for two protein classes, each with several corresponding protein indices, that were each expressed in the HEK293 cell line. As shown, FIGS. 12A-12C include an identification of the metric/condition utilized for each respective protein variant where applicable. In particular, as shown in FIGS. 12A-12C, each respective variant has an associated metric and/or condition where applicable that is utilized. Additionally, As shown in tables 12A-12C, four of the five VHH4 proteins have at least one variant with a 2-fold or more increase in protein expression relative to the wild type/reference sequence, demonstrating the successful performance of the high-yield gene sequence prediction system. As further shown in the table of FIGS. 12A-12C, all of the eight mAb proteins have at least one variant with a 2-fold or more increase in protein expression relative to the wild type/reference sequence, demonstrating the successful performance of the high-yield gene sequence prediction system. In fact, five of the eight proteins had one or more variants exhibit at least a 10-fold increase in protein expression, with three proteins having one or more variants with at least a 20-fold increase in protein expression, and one particular protein having five variants reach at least a 100-fold increase in protein expression.

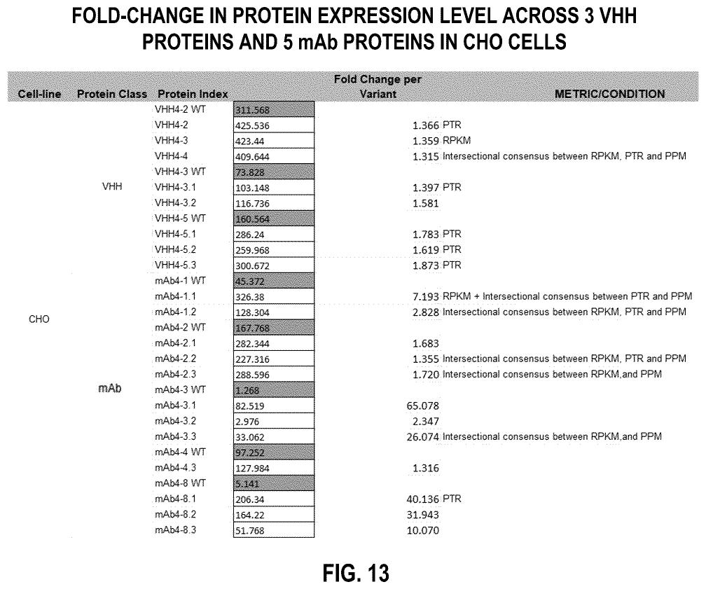

FIG. 13 includes a table that shows the fold-change in yield/expression for two protein classes, each with several corresponding proteins, that were each expressed in the CHO cell line. As shown, FIG. 13 includes an identification of the metric/condition utilized for each respective protein variant where applicable. In particular, as shown in FIG. 13, each respective variant has an associated metric and/or condition where applicable that is utilized. Additionally, As shown in the table of FIG. 13, three of the five mAb proteins have at least one variant with a 7-fold or more increase in protein expression relative to the wild type/reference sequence, demonstrating the successful performance of the high-yield gene sequence prediction system. In fact, two proteins had two of their variants exhibit at least a 26-fold increase in protein expression, with one variant even reaching a 65-fold increase in protein expression. As shown in the table of FIG. 13, the three VHH4 proteins have at least one variant with a 1.3-fold or more increase in protein expression relative to the wild type/reference sequence, demonstrating the successful performance of the high-yield gene sequence prediction system.

### CLAUSES

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed, or elements thereof can be implemented in the form of a computer product, including a non-transitory computer readable storage medium with computer usable program code for performing the method steps indicated. Furthermore, one or more implementations and clauses of the technology disclosed, or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:
1. A computer-implemented method, including:
generating high-yield training datasets by excluding those genetic sequences that do not satisfy at least one abundance condition, at least one stability condition, at least one expression condition, and/or at least one translation efficiency condition; and
training at least one model on the high-yield training datasets to generate high-yield genetic sequences.
2. The method of clause 1, wherein the abundance condition is mRNA abundance.
3. The method of clause 1, wherein the abundance condition is protein abundance.
4. The method of clause 1, wherein the stability condition is mRNA stability.
5. The method of clause 1, wherein the stability condition is protein stability.
6. The method of clause 1, wherein the expression condition is stable/consistent expression.
7. The method of clause 1, wherein the expression condition is ubiquitous expression.
8. The method of clause 2, wherein the mRNA abundance is determined by Reads Per Kilobase Million (RPKM)-based thresholding, Fragments Per Kilobase of Transcript Per Million (FPKM)-based thresholding, and/or Transcripts Per Kilobase Million (TPM)-based thresholding.
9. The method of clause 8, wherein the RPKM-based thresholding excludes from the high-yield training datasets those genetic sequences that do not meet a RPKM threshold, the FPKM-based thresholding excludes from the high-yield training datasets those genetic sequences that do not meet a FPKM threshold, and/or the TPM-based thresholding excludes from the high-yield training datasets those genetic sequences that do not meet a TPM threshold.
10. The method of clause 9, wherein the mRNA abundance is determined by an intersectional consensus of the RPKM-based thresholding, the FPKM-based thresholding, and/or the TPM-based thresholding, wherein the intersectional consensus selects those genetic sequences that overlap between each of the RPKM-based thresholding, the FPKM-based thresholding, and/or the TPM-based thresholding.
11. The method of clause 10, wherein the mRNA abundance is determined by a union consensus of the RPKM-based thresholding, the FPKM-based thresholding, and/or the TPM-based thresholding, wherein the union consensus selects those genetic sequences that merge at least two of the RPKM-based thresholding, the FPKM-based thresholding, and/or the TPM-based thresholding.
12. The method of clause 3, wherein the protein abundance is determined by Parts Per Million (PPM)-based thresholding, Yield Measurement-based thresholding, and/or Titer-based thresholding.
13. The method of clause 12, wherein the PPM-based thresholding excludes from the high-yield training datasets those genetic sequences that do not meet a PPM threshold, the Yield Measurement-based thresholding excludes from the high-yield training datasets those genetic sequences that do not meet a yield measurement threshold, and/or the titer-based thresholding excludes from the high-yield training datasets those genetic sequences that do not meet a titer threshold.
14. The method of clause 13, wherein the protein abundance is determined by an intersectional consensus of the PPM-based thresholding, the Yield Measurement-based thresholding, and/or the titer-based thresholding, wherein the intersectional consensus selects those genetic sequences that overlap between each of the PPM-based thresholding, the Yield Measurement-based thresholding, and/or the titer-based thresholding.
15. The method of clause 14, wherein the protein abundance is determined by a union consensus of the PPM-based thresholding, the Yield Measurement-based thresholding, and/or the titer-based thresholding, wherein the union consensus selects those genetic sequences that merge at least two of the PPM-based thresholding, the Yield Measurement-based thresholding, and/or the titer-based thresholding.
16. The method of clause 4, wherein the mRNA stability is determined by mRNA half-life-based thresholding, and/or degradation rates-based thresholding.
17. The method of clause 16, wherein the mRNA half-life-based thresholding excludes from the high-yield training datasets those genetic sequences that do not meet an mRNA half-life threshold and/or the degradation rates-based thresholding excludes from the high-yield training datasets those genetic sequences that do not meet a degradation threshold.
18. The method of clause 17, wherein the mRNA stability is determined by an intersectional consensus of the mRNA half-life-based thresholding and/or the degradation rates-based thresholding, wherein the intersectional consensus selects those genetic sequences that overlap between each of the mRNA half-life-based thresholding and/or the degradation rates-based thresholding.
19. The method of clause 18, wherein the mRNA stability is determined by a union consensus of the mRNA half-life-based thresholding and/or the degradation rates-based thresholding, wherein the union consensus selects those genetic sequences that merge at least two of the mRNA half-life-based thresholding and/or the degradation rates-based thresholding.
20. The method of clause 5, wherein the protein stability is determined by protein half-life-based thresholding, and/or degradation rates-based thresholding.
21. The method of clause 20, wherein the protein half-life-based thresholding excludes from the high-yield training datasets those genetic sequences that do not meet a protein half-life threshold and/or the degradation rates-based thresholding excludes from the high-yield training datasets those genetic sequences that do not meet a degradation threshold.
22. The method of clause 21, wherein the protein stability is determined by an intersectional consensus of the protein half-life-based thresholding and/or the degradation rates-based thresholding, wherein the intersectional consensus selects those genetic sequences that overlap between each of the protein half-life-based thresholding and/or the degradation rates-based thresholding.
23. The method of clause 22, wherein the protein stability is determined by a union consensus of the protein half-life-based thresholding and/or the degradation rates-based thresholding, wherein the union consensus selects those genetic sequences that merge at least two of the protein half-life-based thresholding and/or the degradation rates-based thresholding.
24. The method of clause 6, wherein the stable/consistent expression is determined by Housekeeping genes-based thresholding and/or Collagen-based thresholding.
25. The method of clause 7, wherein the ubiquitous expression is determined by Housekeeping genes-based thresholding and/or Collagen-based thresholding.
26. The method of clause 1, wherein the translation efficiency condition is determined by Protein-to-mRNA Ratio (PTR)-based thresholding, Protein Per Transcript (PPT)-based thresholding, and/or Ribosomal Profiling-based thresholding.
27. The method of clause 26, wherein the PTR-based thresholding excludes from the high-yield training datasets those genetic sequences that do not meet a PTR threshold, the PPT-based thresholding excludes from the high-yield training datasets those genetic sequences that do not meet a PPT threshold, and/or the Ribosomal Profiling-based thresholding excludes from the high-yield training datasets those genetic sequences that do not meet a ribosomal profiling threshold.
28. The method of clause 27, wherein the translation efficiency condition is determined by an intersectional consensus of the PTR-based thresholding, the PPT-based thresholding, and/or the Ribosomal Profiling-based thresholding, wherein the intersectional consensus selects those genetic sequences that overlap between each of the PTR-based thresholding, the PPT-based thresholding, and/or the Ribosomal Profiling-based thresholding.
29. The method of clause 28, wherein the translation efficiency condition is determined by a union consensus of the PTR-based thresholding, the PPT-based thresholding, and/or the Ribosomal Profiling-based thresholding, wherein the union consensus selects those genetic sequences that merge at least one of the PTR-based thresholding, the PPT-based thresholding, and/or the Ribosomal Profiling-based thresholding.
30. The method of clause 1, further including generating the high-yield training datasets based on an intersectional consensus of the abundance condition, the stability condition, and/or the expression condition, wherein the intersectional consensus selects those genetic sequences that overlap between each of the abundance condition, the stability condition, the expression condition, and/or the translation efficiency condition.
31. The method of clause 1, further including generating the high-yield training datasets based on a union consensus of the abundance condition, the stability condition, and/or the expression condition, wherein the union consensus selects those genetic sequences that merge at least one of the abundance condition, the stability condition, the expression condition, and/or the translation efficiency condition.
32. The method of claim 1, wherein each genetic sequence includes at least one predicted value, and wherein the high-yield training datasets are generated by excluding those genetic sequences where the at least one predicted value does not satisfy at least one abundance condition, at least one stability condition, at least one expression condition, and/or at least one translation efficiency condition.
33. The method of claim 32, wherein the at least one predicted value is inferred by one or more regressors.
34. The method of claim 1, wherein the genetic sequences include codon sequences, and wherein the high-yield genetic sequences include high-yield codon sequences.

1. A computer-implemented method, including:
generating high-yield training datasets by excluding those genetic sequences that do not satisfy at least one abundance condition, at least one stability condition, at least one expression condition, and/or at least one translation efficiency condition; and
training at least one model on the high-yield training datasets to generate high-yield genetic sequences.

2. The method of claim 1, wherein the abundance condition is mRNA abundance.

3. The method of claim 1, wherein the abundance condition is protein abundance.

4. The method of claim 1, wherein the stability condition is mRNA stability.

5. The method of claim 1, wherein the stability condition is protein stability.

6. The method of claim 1, wherein the expression condition is stable/consistent expression.

7. The method of claim 1, wherein the expression condition is ubiquitous expression.

8. The method of claim 2, wherein the mRNA abundance is determined by Reads Per Kilobase Million (RPKM)-based thresholding, Fragments Per Kilobase of Transcript Per Million (FPKM)-based thresholding, and/or Transcripts Per Kilobase Million (TPM)-based thresholding.

9. The method of claim 8, wherein the RPKM-based thresholding excludes from the high-yield training datasets those genetic sequences that do not meet a RPKM threshold, the FPKM-based thresholding excludes from the high-yield training datasets those genetic sequences that do not meet a FPKM threshold, and/or the TPM-based thresholding excludes from the high-yield training datasets those genetic sequences that do not meet a TPM threshold.

10. The method of claim 9, wherein the mRNA abundance is determined by an intersectional consensus of the RPKM-based thresholding, the FPKM-based thresholding, and/or the TPM-based thresholding, wherein the intersectional consensus selects those genetic sequences that overlap between each of the RPKM-based thresholding, the FPKM-based thresholding, and/or the TPM-based thresholding.

11. The method of claim 10, wherein the mRNA abundance is determined by a union consensus of the RPKM-based thresholding, the FPKM-based thresholding, and/or the TPM-based thresholding, wherein the union consensus selects those genetic sequences that merge at least two of the RPKM-based thresholding, the FPKM-based thresholding, and/or the TPM-based thresholding.

12. The method of claim 3, wherein the protein abundance is determined by Parts Per Million (PPM)-based thresholding, Yield Measurement-based thresholding, and/or Titer-based thresholding.

13. The method of claim 12, wherein the PPM-based thresholding excludes from the high-yield training datasets those genetic sequences that do not meet a PPM threshold, the Yield Measurement-based thresholding excludes from the high-yield training datasets those genetic sequences that do not meet a yield measurement threshold, and/or the titer-based thresholding excludes from the high-yield training datasets those genetic sequences that do not meet a titer threshold.

14. The method of claim 13, wherein the protein abundance is determined by an intersectional consensus of the PPM-based thresholding, the Yield Measurement-based thresholding, and/or the titer-based thresholding, wherein the intersectional consensus selects those genetic sequences that overlap between each of the PPM-based thresholding, the Yield Measurement-based thresholding, and/or the titer-based thresholding.

15. The method of claim 14, wherein the protein abundance is determined by a union consensus of the PPM-based thresholding, the Yield Measurement-based thresholding, and/or the titer-based thresholding, wherein the union consensus selects those genetic sequences that merge at least two of the PPM-based thresholding, the Yield Measurement-based thresholding, and/or the titer-based thresholding.

16. The method of claim 4, wherein the mRNA stability is determined by mRNA half-life-based thresholding, and/or degradation rates-based thresholding.

17. The method of claim 16, wherein the mRNA half-life-based thresholding excludes from the high-yield training datasets those genetic sequences that do not meet an mRNA half-life threshold and/or the degradation rates-based thresholding excludes from the high-yield training datasets those genetic sequences that do not meet a degradation threshold.

18. The method of claim 17, wherein the mRNA stability is determined by an intersectional consensus of the mRNA half-life-based thresholding and/or the degradation rates-based thresholding, wherein the intersectional consensus selects those genetic sequences that overlap between each of the mRNA half-life-based thresholding and/or the degradation rates-based thresholding.

19. The method of claim 18, wherein the mRNA stability is determined by a union consensus of the mRNA half-life-based thresholding and/or the degradation rates-based thresholding, wherein the union consensus selects those genetic sequences that merge at least two of the mRNA half-life-based thresholding and/or the degradation rates-based thresholding.

20. The method of claim 5, wherein the protein stability is determined by protein half-life-based thresholding, and/or degradation rates-based thresholding.

21. The method of claim 20, wherein the protein half-life-based thresholding excludes from the high-yield training datasets those genetic sequences that do not meet a protein half-life threshold and/or the degradation rates-based thresholding excludes from the high-yield training datasets those genetic sequences that do not meet a degradation threshold.

22. The method of claim 21, wherein the protein stability is determined by an intersectional consensus of the protein half-life-based thresholding and/or the degradation rates-based thresholding, wherein the intersectional consensus selects those genetic sequences that overlap between each of the protein half-life-based thresholding and/or the degradation rates-based thresholding.

23. The method of claim 22, wherein the protein stability is determined by a union consensus of the protein half-life-based thresholding and/or the degradation rates-based thresholding, wherein the union consensus selects those genetic sequences that merge at least two of the protein half-life-based thresholding and/or the degradation rates-based thresholding.

24. The method of claim 6, wherein the stable/consistent expression is determined by Housekeeping genes-based thresholding and/or Collagen-based thresholding.

25. The method of claim 7, wherein the ubiquitous expression is determined by Housekeeping genes-based thresholding and/or Collagen-based thresholding.

26. The method of claim 1, wherein the translation efficiency condition is determined by Protein-to-mRNA Ratio (PTR)-based thresholding, Protein Per Transcript (PPT)-based thresholding, and/or Ribosomal Profiling-based thresholding.

27. The method of claim 26, wherein the PTR-based thresholding excludes from the high-yield training datasets those genetic sequences that do not meet a PTR threshold, the PPT-based thresholding excludes from the high-yield training datasets those genetic sequences that do not meet a PPT threshold, and/or the Ribosomal Profiling-based thresholding excludes from the high-yield training datasets those genetic sequences that do not meet a ribosomal profiling threshold.

28. The method of claim 27, wherein the translation efficiency condition is determined by an intersectional consensus of the PTR-based thresholding, the PPT-based thresholding, and/or the Ribosomal Profiling-based thresholding, wherein the intersectional consensus selects those genetic sequences that overlap between each of the PTR-based thresholding, the PPT-based thresholding, and/or the Ribosomal Profiling-based thresholding.

29. The method of claim 28, wherein the translation efficiency condition is determined by a union consensus of the PTR-based thresholding, the PPT-based thresholding, and/or the Ribosomal Profiling-based thresholding, wherein the union consensus selects those genetic sequences that merge at least one of the PTR-based thresholding, the PPT-based thresholding, and/or the Ribosomal Profiling-based thresholding.

30. (canceled)

31. (canceled)

32. (canceled)

33. (canceled)

34. (canceled)
